(19) United States
(12) Patent Application Publication
(10) Pub. No.: US 2008/0269666 A1
(43) Pub. Date: Oct. 30, 2008

(54) Microneedles and Methods for Microinfusion

(75) Inventors: Ping M. Wang, Norcross, GA (US); Mark R. Prausnitz, Atlanta, GA (US); Wijaya Martanto, Beaverton, OR (US)

Correspondence Address: SUTHERLAND ASBILL & BRENNAN LLP, 999 PEACHTREE STREET, N.E., ATLANTA, GA, 30309, US

(73) Assignee: GEORGIA TECH RESEARCH CORPORATION, Atlanta, GA (US)

(21) Appl. No.: 11/914,366

(22) PCT Filed: May 25, 2006
(86) PCT No.: PCT/US06/20546

§ 371 Date: Nov. 13, 2007

Related U.S. Application Data
(60) Provisional application No. 60/684,273, filed on May 25, 2005.
(60) Provisional application No. 60/691,863, filed on Jun. 18, 2005.

(51) Int. Cl. A61N 1/30; A61F 2/00; A61K 35/00; A61K 35/76; A61K 39/00; A61M 1/00; A61N 7/00; A61K 38/28; A61K 38/47; A61M 5/32

(52) U.S. Cl. 604/21; 424/422; 424/93.1; 424/93.6; 424/184.1; 514/3; 424/94.62; 424/94.6; 604/506; 604/22; 604/173; 604/60; 604/151

(57) ABSTRACT

Methods and devices are provided for delivering a drug to or withdrawing a fluid from a biological tissue, such the skin, sclera, cornea, and conjunctiva. One method includes the steps of inserting at least one microneedle into the biological tissue; partially retracting the at least one microneedle from the tissue; and then delivering at least one drug formulation into the biological tissue via the partially retracted at least one microneedle. The microneedle deforms and penetrates the biological tissue during the insertion step, and the retraction step at least partially relaxes the tissue deformation while maintaining at least part of the tissue penetration, facilitating drug delivery or fluid withdrawal.

### CROSS REFERENCE TO RELATED APPLICATIONS

This application claims benefit of U.S. Provisional Application No. 60/691,863, filed Jun. 17, 2005, and U.S. Provisional Application No. 60/684,273, filed May 25, 2005. Those applications are incorporated herein by reference in their entirety.

### STATEMENT REGARDING FEDERALLY SPONSORED RESEARCH OR DEVELOPMENT

This invention was made with U.S. government support under Contract No. 8 RO1 EB00260-03 awarded by the National Institute of Health. The U.S. government has certain rights in the invention.

### BACKGROUND OF THE INVENTION

This invention is generally in the field of transdermal therapies, and more particularly to the use of microneedles for drug delivery.

Clinical and research applications require delivery of drug or other compounds into skin for local dermatological effects or systemic effects after absorption into the bloodstream via dermal capillaries. Such compounds are usually delivered to the skin by passive mechanisms, where a topical formulation or patch is applied to the skin surface and the drug compound diffuses into the skin across stratum corneum. Because the stratum corneum's barrier properties severely limit passive delivery of most drugs, and especially macromolecules and microparticles, intradermal injection using a hypodermic needle is an effective alternative. However, the pain and inconvenience of intradermal injection generally limits self-administration by patients or other uses outside the clinic or laboratory. Moreover, the relatively large size of hypodermic needles and their relatively poorly controlled manual insertion into skin makes targeted delivery to sites within skin difficult.

Infusion pumps are used for many clinical applications, including intravenous, epidural, and subcutaneous delivery of analgesics and anesthetics, antibiotics, cardiovascular drugs, and insulin. Drug delivery via infusion reduces the plasma drug concentration fluctuation associated with oral delivery and the slow onset and long depot effect associated with transdermal patch delivery. Infusion pumps are commonly used when continuous, intermittent or pulsatile delivery of drug is needed. They also provide an alternative for patients intolerant to oral administration and can be programmed to achieve special delivery profiles. The use of infusion pumps outside the clinical setting has been limited by the bulky size and expensive cost of such devices, as well as by low patient compliance due to the inconvenience of an indwelling catheter that has a relatively large infusion set and the expertise required to properly use it (see Liebl, Diabetes Metab Res Rev 18:S36-S41 (2002); Moulin & Kreeft, Lancet 337:465-68 (1991)).

One conventional drug delivery device which attempts to address some of the foregoing issues is a compact, disposable device that incorporates a relatively short, 5-mm long hypodermic needle. It has been shown to continuously and subcutaneously infuse drug solutions, such as heparin to prevent thrombosis and morphine sulfate for management of cancer pain (see Meehan, et al., J Control Release 46:107-16 (1997); Lynch, et al., J Pain Symptom Manage 19:348-56 (2000)). The type of device delivers drug from a pressure-driven reservoir through the needle into the skin. Despite its minimally invasive approach, local erythema, edema, and contact dermatitis at the injection site have been reported with the use of this device (Jolanki, et al., Contact Dermatitis 45:36-37 (2001)).

Microneedles were originally designed to increase skin permeability for patch-based delivery by diffusion. Solid microneedles have been shown to increase transdermal transport by orders of magnitude in vitro for a variety of compounds (see Henry, et al., J Pharm Sci 87:922-25 (1998); McAllister, et al., Proc Natl Acad Sci USA 100:13755-60 (2003); Chabri, et al., Br J Dermatol 150:869-77 (2004)). For example, in vivo studies have demonstrated delivery of insulin (see Martanto, et al., Pharm Res 21:947-52 (2004)), oligonucleotides (see Lin, et al., Pharm Res 18: 1789-93 (2001)), human growth hormone (see Cormier & Daddona, Modified-Release Drug Delivery Technology pp. 589-98), and desmopressin (see Cormier, et al., J Control Release 97:503-11 (2004)), as well as ovalbumin (see Matriano, et al., Pharm Res 19:63-70 (2002)), DNA (see Mikszta, et al., Nat Med 8: 415-19 (2002)) and anthrax vaccines (see Mikszta, et al., J Infect Dis 191:278-88 (2005)). Human pain studies have shown that insertion of microneedles can be painless and does not cause skin irritation (see Mikszta, et al., Nat Med 8:415-19 (2002); Kaushik, et al., Anesth Analg 92: 502-04 (2001)).

Although significant attention has been given to fabrication of both solid and hollow microneedles, most drug delivery studies have employed just solid microneedles used either to pierce holes in the skin as a pretreatment before application of a transdermal patch or coated with drug that dissolves off the microneedles upon insertion into the skin. Microneedles containing a hollow bore have also been made using a variety of different microfabrication approaches (see McAllister, et al., Annu Rev Biomed Eng 2:289-313 (2000); Reed & Lye, Proc IEEE 92: 56-75 (2004)). Hollow microneedles have received less attention in part because they are harder to use. For example, hollow microneedles are inherently weaker than solid microneedles and therefore have additional constraints on needle design and insertion methods (see Davis, et al., J Biomech 37:1155-63 (2004)). Placement of the bore opening at the needle tip reduces needle tip sharpness and makes insertion into skin more difficult. Moreover, flow through the bore of hollow microneedles is also difficult to achieve.

Despite these difficulties, hollow microneedles have been shown to deliver pharmacologically active insulin to diabetic rats (see Gardeniers, et al., J MEMS 12: 855-62 (2003); McAllister, et al., Proc Natl Acad Sci USA 100:13755-60 (2003)) and to passively deliver insulin by diffusion to diabetic rats (see Davis, et al., IEEE Trans Biomed Eng 52:909-15 (2005)). Active injection using hollow microneedles coupled to a syringe or pump has been demonstrated for administration of chemical stimuli into brain tissue in vivo (Chen, et al., IEEE Trans Biomed Eng 44: 760-69 (1997)) and injection of fluorescent dye into chicken thigh in vitro (see Stoeber & Liepmann, Proceedings of Solid-State Sensor and Actuator Workshop, Transducers Research Foundation (2002)).

It therefore would be desirable to provide a microneedle device and method for the controlled microinjection of drugs into skin using hollow microneedles. It would be desirable to provide methods and devices that avoid the limitations and disadvantages associated with the use of conventional hypodermic needles and conventional infusion pumps for controlled drug delivery.

It also would be desirable to provide additional and improved microneedle devices and techniques for precise microinjection at controlled locations within the skin and at controlled rates of fluid flow. For instance, the spatial targeting of microinjection is desirable for clinical applications that seek to target vaccines to epidermal Langerhans cells, systemic drugs to the superficial dermal capillary bed, or local drugs to the deeper dermis to minimize systemic absorption. Spatially targeted delivery is also desirable for applications that seek to selectively deliver macromolecules, particles used as drug carriers, or possibly cells to different regions of the skin.

### SUMMARY OF THE INVENTION

In one aspect, methods are provided for delivering a drug to a biological tissue. Examples of suitable biological tissues include skin, sclera, cornea, and conjunctiva. The methods include the steps of inserting at least one microneedle into the biological tissue; partially retracting the at least one microneedle from the tissue; and then delivering at least one drug formulation into the biological tissue via the partially retracted at least one microneedle. Preferably, the at least one microneedle deforms and penetrates the biological tissue during the insertion step, and the retraction step at least partially relaxes the tissue deformation while maintaining at least part of the tissue penetration. In one embodiment, the microneedle is rotated about its longitudinal axis during the insertion step, during the partial retraction step, or during both steps. For example, the insertion and retraction steps may be performed in a controlled fashion calibrated by the number of rotations of the microneedle. In one embodiment, the microneedle is vibrated during the insertion step, during the partial retraction step, or during both steps. The microneedle may be retracted a distance that is between 10% and 90% of the initial insertion depth. The initial insertion depth may be between 200 μm and 5000 μm. In one embodiment, the method uses an array of two or more of the microneedles.

In a preferred embodiment, the microneedle is hollow and the drug formulation is fluid and flows through the hollow microneedle into the biological tissue. In one embodiment, the microneedle has at least one bore and a beveled tip. The fluid drug formulation typically should be driven through the at least one microneedle in a manner effective to drive the fluid drug formulation into the biological tissue. The fluid drug formulation may be driven by diffusion, capillary action, a mechanical pump, electroosmosis, electrophoresis, convection, magnetic field, ultrasound, or a combination thereof.

The drug formulation may include macromolecules, microparticles, nanoparticles, cells, viruses, or a combination thereof. It may include a therapeutic agent, a prophylactic agent, or a diagnostic agent. Examples include vaccines and insulin.

In various embodiments, the methods may be adapted to deliver the drug formulation specifically to the epidermis, dermis, or subcutaneous tissue.

In another aspect, which may be used separately or in conjunction with the foregoing aspect, methods are provided for transdermal delivery of drug to a patient which includes the steps of inserting at least one microneedle into the skin of the patient; delivering at least one drug formulation into the skin via the at least one microneedle; and co-administering at least one compound that alters the microstructure of the skin in combination with the delivery of the at least one drug formulation. In one embodiment, the compound comprises hyaluronidase, collagenase, or another enzyme.

In still another aspect, methods are provided for microinfusion of a fluid into a biological tissue which include the steps of inserting at least one hollow microneedle into the biological tissue; and driving a fluid drug formulation from a fluid reservoir through the hollow microneedle into the biological tissue, wherein deformation of the biological tissue is reduced by performing the insertion step is performed using microneedle velocity, microneedle vibration, microneedle rotation, tissue stretching, or a combination thereof.

In a further aspect, methods are provided for fluid extraction from a biological tissue which include the steps of inserting at least one hollow microneedle into the biological tissue; partially retracting the at least one microneedle from the tissue; and withdrawing at least one biological fluid from the biological tissue via the partially retracted at least one microneedle.

In another aspect, a microneedle device is provided for delivery of a fluid drug formulation to or withdrawal of a fluid from to a biological tissue. The device includes at least one microneedle having a tip portion for penetrating skin or another biological tissue; means for controllably inserting the at least one microneedle into the biological tissue, deforming the biological tissue; and means for controllably retracting the at least one microneedle partially from the tissue to at least partially relax the tissue deformation. The device may further include means for rotating the at least one microneedle about its longitudinal axis during the insertion step, during the partial retraction step, or during both steps. The device also may further include means for vibrating the at least one microneedle. The device preferably includes a fluid reservoir storing at least one fluid drug formulation to be delivered into the biological tissue.

In one embodiment, the microneedle device includes a holder with a bottom surface for contacting the biological tissue; an opening in the bottom surface allowing the at least one microneedle to pass through; and an insert disposed inside the holder, the insert having a through bore configured to receive the at least one microneedle so positioned to pass through the opening. The movement of the insert along the longitudinal axis may be effectuated by a mechanical coupling element attached to the insert. For instance, the mechanical coupling element may include a gear for coupling to another gear, a motor, or a micromotor, or may be manually driven.

In one preferred embodiment, the microneedle is hollow and has a beveled tip. The microneedle may be formed of a coated or uncoated metal, silicon, glass, or ceramic. In one embodiment, the microneedle may include or be formed of a polymer.

In various embodiments, the means for controllably inserting the microneedle is adapted to provide a maximum insertion depth into the biological tissue between 200 μm and 5 mm, for example, between 500 and 1500 μm.

In one embodiment, the microneedle device includes at least one pump device for pumping the drug from the fluid source, through the tip end of the microneedle, and into the biological tissue.

### BRIEF DESCRIPTION OF THE DRAWINGS

FIGS. 1A-B are micrographs showing front (FIG. 1A) and side (FIG. 1B) views of one embodiment of a hollow, glass microneedle.

FIGS. 2A-B are micrographs showing histological section of human cadaver skin untreated (FIG. 2A) and following piercing with, and subsequent removal of, a hollow microneedle in vitro (FIG. 2B).

FIGS. 3A-B are micrographs showing top (FIG. 3A) and bottom (FIG. 3B) surfaces of human cadaver skin after infusion of sulforhodamine solution using a hollow microneedle in vitro. The site of microneedle penetration is shown by the arrow (FIG. 3A).

FIGS. 4A-E are graphs showing the effect of insertion depth and retraction distance on flow rate into human cadaver skin in vitro.

FIG. 5 is a graph showing the effect of pressure on flow rate into human cadaver skin.

FIG. 6 is a graph showing the effect of tip bevel on flow rate into human cadaver skin.

FIG. 7 is a graph showing the effect of tip opening size on flow rate into human cadaver skin.

FIG. 8 is a graph showing the effect of hyaluronidase on flow rate into human cadaver skin.

FIG. 9 is a graph showing cumulative infusion volume during infusion into human cadaver skin over time, with and without microneedle retraction.

FIG. 10 is a graph showing the effect of skin on flow rate through microneedles.

FIGS. 11A-B are micrographs of hollow, glass microneedles. FIG. 11A shows a microneedle with a bore opening at a blunt tip, and FIG. 11B shows a microneedle with a side-opening bore at a beveled tip. Bar=100 μm.

FIGS. 12A-D are images of multi-needle glass and polymer microneedle arrays. FIGS. 12A and 12B show glass microneedles assembled into an array using epoxy. FIGS. 12C and 12D respectively show a poly-glycolide microneedles and a polylactide-co-glycolide microneedle array molded from glass microneedle masters. Unlabeled bars=100 μm.

FIGS. 13A-B are process diagrams showing microscopic methods used to image microinjection into skin. FIG. 13A shows a scheme of the direct-imaging method using a video-microscopy system focused on a cryostat microtome to image the dyed area in each skin cross-section, and FIG. 13B shows a scheme of the in situ imaging of transdermal microinjection in real time using skin placed on the stage of an inverted microscope.

FIGS. 14A-D are micrographs showing microneedle track pathways when inserted into hairless rat skin in vivo. FIG. 14A shows a hole in skin imaged in situ on the skin surface of an anesthetized hairless rat after insertion and removal of a microneedle. FIG. 14B is a sequence of images showing H&E-stained histological sections taken parallel to the surface of in vitro hairless rat skin surface after piercing with a microneedle. FIG. 14C is a sequence of images showing H&E-stained histological sections taken perpendicular to the surface of in vitro hairless rat skin after piercing with a microneedle and injecting tissue-marking dye. FIG. 14D is a set of images showing areas of dye staining determined by image processing of the upper set of histological sections.

FIGS. 15A-B are micrographs showing highly targeted microinjection at various depths within the dermis and epidermis, H&E-stained histological sections of hairless rat skin microinjected in vivo with tissue marking dye at three controlled depths (arrows) in the dermis (FIG. 15A, scale bar=100 μm) and localized within the epidermis (FIG. 15B, scale bar=50 μm).

FIGS. 16A-B are fluorescence micrographs showing calcein microinjection into hairless rat skin in vitro at 10 psi, with and without partial microneedle retraction.

FIG. 17A-B are micrographs showing hairless rat skin microinjected in vivo with FITC-insulin. Histological sections are shown using brightfield microscopy with H&E staining (FIG. 17A) and fluorescence microscopy showing injection and distribution of insulin in the skin (FIG. 17B). Scale bar=100 μm.

FIG. 18 is a graph showing changes in blood glucose level in diabetic hairless rats before, during, and after a 30-min. infusion from a microneedle.

FIGS. 19A-B are micrographs showing hairless rat skin microinjected in vivo with polymeric particles and cells. FIG. 19A shows a brightfield micrograph of H&E-stained histological section following microinjection with 2.8-μm polymeric microspheres. (Inset shows magnified view of microspheres in skin.) FIG. 19B shows fluorescence micrograph of histological section after microinjection of fluorescence-labeled Caco-2 human intestinal epithelial cells.

FIGS. 20A-B are fluorescence micrographs of histological sections after microinjection of 2.5-μm fluorescent microspheres into hairless rat skin in vivo under pressures ranging from 2.5 to 20 psi via the same needle and loading for the same time periods. The volume and depth of injection increased with increasing pressure.

FIGS. 21A-B are micrographs showing the effect of vibration of microneedle arrays during insertion to increase microinjection. FIG. 21A shows intracutaneous staining imaged in situ at the skin surface when the needle array was inserted without vibration for 10 min, and FIG. 21B show intracutaneous staining imaged in situ at the skin surface when the needle array was vibrated during and for 1 min. after insertion.

FIG. 22 shows a series of micrographs of histological sections of human cadaver skin pierced with a hollow microneedle in vitro, shown at low (A) and high (B) magnification.

FIG. 23 is a cross-sectional view of one embodiment of microneedle device with holder and insert for controlling microneedle insertion.

FIG. 24 shows in cross-sectional views the sequence of one embodiment of the delivery process, in which single hollow microneedle is inserted into skin, partially retracted, fluid is delivered through the microneedle, and then the microneedle is withdrawn completely.

### DETAILED DESCRIPTION OF THE PREFERRED EMBODIMENTS

Microneedle devices and methods of use have been developed for delivering substances into biological tissues. Importantly, with the present methods and devices, the depth of insertion into the skin or other biological tissue is precisely controlled, enabling optimum targeting of location and enhanced control of flow of fluid to be delivered.

In one aspect, it has been discovered that infusion of a drug formulation, or fluid withdrawal, through hollow microneedles can be greatly enhanced by partial retraction of the microneedle from the biological tissue following initial insertion and before/during fluid delivery. Advantageously, this method provides one approach for mitigating the deformation of the biological tissue that may occur when the microneedle is inserted into the tissue, deformation which otherwise can degrade control of fluid delivery through the needle. Significantly, withdrawal of the microneedle was discovered not to leave a cavity that would be filled with the fluid drug, but rather mitigates deformation which facilitate flow of the fluid directly into the tissues.

In another aspect, methods of transdermal delivery of drug to a patient have developed that include the steps of inserting one or more microneedles into the skin of the patient; delivering at least one drug formulation into the skin via the one or more microneedles; and co-administering at least one compound that alters the microstructure of the skin in combination with the delivery of the at least one drug formulation.

The microneedles may be coupled with a micropump to make a wearable infusion device that is highly patient friendly and can serve as a potential replacement for conventional hypodermic needles and infusion sets. Microneedles are expected to be safe, because they are minimally invasive devices that typically are inserted only into skin's superficial layers and are typically bloodless and painless. Microneedles are also expected to be effective, as a hybrid between transdermal patches and conventional injection/infusion systems.

As used herein, the term “biological tissue” includes essentially any cells, tissue, or organs, including the skin or parts thereof, mucosal tissues, vascular tissues, lymphatic vessels, ocular tissues (e.g., cornea, conjunctiva, sclera), and cell membranes. The biological tissue can be in humans or other types of animals (particularly mammals), as well as in plants, insects, or other organisms, including bacteria, yeast, fungi, and embryos. Human skin and sclera are biological tissues of particular use with the present devices and methods.

As used herein, the terms “comprise,” “comprising,” “include,” and “including” are intended to be open, non-limiting terms, unless the contrary is expressly indicated.

### The Methods of Microinfusion or Delivery

The amount of drug delivered within the tissue may be controlled, in part, by the type of microneedle used and how it is used. In a preferred embodiment, a hollow microneedle is inserted into the biological tissue and partially retracted from the tissue before or during the fluid delivery period. There are various methods to control the insertion depth. In one preferred embodiment, the microneedles are designed to have a length equal to the desired penetration depth. In another preferred embodiment, the microneedles are designed to have a length longer than the desired penetration depth, but the microneedles are only inserted part way into the tissue. Partial insertion may be controlled by the mechanical properties of the tissue, which bends and dimples during the microneedle insertion process. In this way, as the microneedle is inserted into the tissue, its movement partially bends the tissue and partially penetrated into the tissue. By controlling the degree to which the tissue bends, the depth of microneedle penetration into the tissue can be controlled.

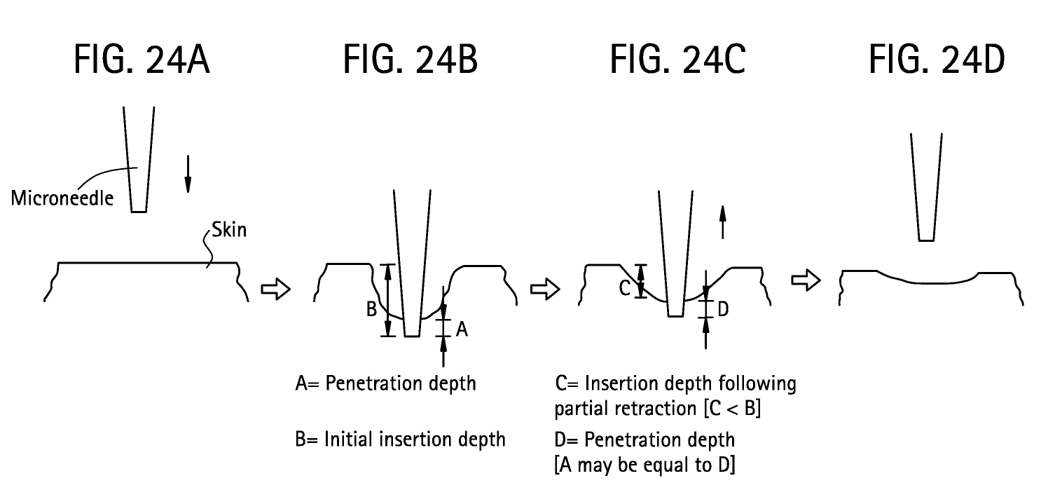

In one aspect, methods are provided for delivering a drug to a biological tissue. The methods include the steps of inserting at least one microneedle into the biological tissue; partially retracting the at least one microneedle from the tissue; and then delivering at least one drug formulation into the biological tissue via the partially retracted at least one microneedle. Preferably, the at least one microneedle deforms and penetrates the biological tissue during the insertion step, and the retraction step at least partially relaxes the tissue deformation while maintaining at least part of the tissue penetration. See FIG. 24.

The initial insertion depth of the microneedle may be between 200 μm and 5000 μm (e.g., more than 250 μm, 500 μm, 800 μm, or 1000 μm, and e.g., less than 4000 μm, 3000 μm, 2500 μm, 2000 μm, 1800 μm, or 1500 μm). In one embodiment, the insertion depth is between 500 and 1500 μm. In suitable embodiments, the one or more microneedles are retracted a distance that is at least 10% of the depth to which the one or more microneedles are initially inserted. In various embodiments, the retraction distance is between 20 and 95% (e.g., between 50 and 90%) of the initial insertion depth.

As used herein, the terms “insertion depth” and the process of “inserting” the microneedle into biological tissue refer to the movement of the microneedle into the surface of the skin, and this depth includes both the distance the tissue is deformed (by the microneedle) and the distance the tissue is penetrated by the microneedle. The term “penetration depth” refers to the non-deformative incursion of the microneedle into the tissue. In other words, insertion depth equals penetration distance plus deformation distance under the tip of the microneedle. See FIG. 24.

In a preferred embodiment, the microneedles are inserted into the tissue using a drilling or vibrating action. In this way, the microneedles can be inserted to a desired depth by, for example, drilling the microneedles a desired number of rotations, which corresponds to a desired depth into the tissue. See, e.g., U.S. Patent Application Publication No. 20050137525 A1 to Wang et al., which is incorporated herein by reference in its entirety. For instance, the one or more microneedles may be rotated about the longitudinal axes of the one or more microneedles during the insertion step, during the partial retraction step, or during both steps. In one embodiment, the one or more microneedles are vibrated during the insertion step, during the partial retraction step, or during both steps. Vibration of the microneedle in essentially any direction or directions may be effective. However, in one embodiment, the vibration is in a direction substantially parallel to the longitudinal axis of the microneedle.

In one embodiment, the microneedle is rotated about its longitudinal axis during the insertion step, during the partial retraction step, or during both steps. For example, the insertion and retraction steps may be performed in a controlled fashion calibrated by the number of rotations of the microneedle. In one embodiment, the microneedle is vibrated during the insertion step, during the partial retraction step, or during both steps. The microneedle may be retracted a distance that is between 10% and 90% of the initial insertion depth. The initial insertion depth may be between 200 μm and 5000 μm. In one embodiment, the method uses an array of two or more of the microneedles.

In a preferred embodiment, the microneedle is hollow and the drug formulation is fluid and flows through the hollow microneedle into the biological tissue. In one embodiment, the microneedle has at least one bore and a beveled tip. The fluid drug formulation typically should be driven through the at least one microneedle in a manner effective to drive the fluid drug formulation into the biological tissue. The fluid drug formulation may be driven by diffusion, capillary action, a mechanical pump, electroosmosis, electrophoresis, convection, magnetic field, ultrasound, or a combination thereof. These driving mechanisms are known and may be readily adapted to the present microneedles and methods.

In various embodiments, the methods may be adapted to deliver the drug formulation specifically to the epidermis, dermis, or subcutaneous tissue.

In one method for transdermal delivery of drug to a patient, the method includes the steps of inserting at least one microneedle into the skin of the patient; delivering at least one drug formulation into the skin via the at least one microneedle; and co-administering at least one compound that alters the microstructure of the skin in combination with the delivery of the at least one drug formulation. Examples of such compounds include solvents such as DMSO or collagen- or hyaluronic acid-degrading enzymes. In preferred embodiments, the at least one compound comprises an enzyme. In a preferred embodiment, the enzyme is hyaluronidase or collagenase or a combination thereof.

In still another aspect, methods are provided for microinfusion of a fluid into a biological tissue which include the steps of inserting at least one hollow microneedle into the biological tissue; and driving a fluid drug formulation from a fluid reservoir through the hollow microneedle into the biological tissue, wherein deformation of the biological tissue is intentionally reduced by performing the insertion step is performed using microneedle velocity, microneedle vibration, microneedle rotation, tissue stretching, or a combination thereof. These methods provide another approach for dealing with the problem of tissue deformation during insertion of the microneedle.

In a further aspect, methods are provided for fluid extraction from a biological tissue which include the steps of inserting at least one hollow microneedle into the biological tissue; partially retracting the at least one microneedle from the tissue; and withdrawing at least one biological fluid from the biological tissue via the partially retracted at least one microneedle.

### The Microneedle Device

In a preferred embodiment, the microneedle device includes at least one microneedle having a tip portion for penetrating skin or another biological tissue; means for controllably inserting the at least one microneedle into the biological tissue; and means for controllably retracting the at least one microneedle partially from the tissue to at least partially relax the tissue deformation. In various embodiments, the means for controllably inserting the microneedle is adapted to provide a maximum insertion depth into the biological tissue between 200 μm and 5000 μm, for example, between 500 and 1500 μm.

The device preferably includes a fluid reservoir storing at least one fluid drug formulation to be delivered into the biological tissue. In one embodiment, the microneedle device includes at least one pump device for pumping the drug from the fluid source, through the tip end of the microneedle, and into the biological tissue.

The device may further include means for rotating the at least one microneedle about its longitudinal axis during the insertion step, during the partial retraction step, or during both steps. See U.S. Patent Application Publication No. 2005/0137525 A1, which is incorporated herein by reference. The device also may further include means for vibrating the at least one microneedle.

In one embodiment, the microneedle device includes a holder with a bottom surface for contacting the biological tissue; an opening in the bottom surface allowing the at least one microneedle to pass through; and an insert disposed inside the holder, the insert having a through bore configured to receive the at least one microneedle so positioned to pass through the opening. The movement of the insert along the longitudinal axis may be effectuated by a mechanical coupling element attached to the insert. For instance, the mechanical coupling element may include a gear for coupling to another gear, a motor, or a micromotor, or may be manually driven.

In one embodiment, the microneedle device includes a substantially planar foundation from which one or more microneedles extend, typically in a direction normal (i.e., perpendicular) to the foundation. The microneedle may be hollow or solid. The microneedle can be porous or non-porous.

Hollow microneedles are typically preferred for use with the present microinfusion methods. As used herein, the term “hollow microneedle” refers to microneedles that have one or more continuous pathways from the base end of the microneedle, through the microneedle and terminating at a point on the microneedle surface that is in communication with the interior of the biological tissue (following microneedle insertion/penetration). These pathways may be in the form of (i) one or more individual bores through the microneedles, (ii) a network of interconnected porosities within the microneedle, (iii) one or more channels along the microneedle surface that are substantially, but not fully surrounded by the microneedle; or (iv) a combination thereof. In one preferred embodiment, the microneedle has a single bore and a beveled tip.

The microneedle can be formed/constructed of different biocompatible materials, including metals, glasses, semi-conductor materials, ceramics, or polymers. Examples of suitable metals include pharmaceutical grade stainless steel, gold, titanium, nickel, iron, gold, tin, chromium, copper, and alloys thereof. In one embodiment, the microneedle is formed of a coated or uncoated metal, silicon, glass, or ceramic. In another embodiment, the microneedle may include or be formed of a polymer. The polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Representative non-biodegradable polymers include polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Biodegradable microneedles can provide an increased level of safety compared to non-biodegradable ones, such that they are essentially harmless even if inadvertently broken off into the biological tissue. This applies whether the microneedles contain molecules for delivery or serve merely a conduit function.

The microneedles may be mass-fabricated using a variety of different methods. Single needles and multi-needle arrays can be made of a variety of different materials. Single, glass microneedles may be produced in various geometries, are physiologically inert, permit easy visualization of fluid flow, and can be fabricated with dimensions similar to those of microfabricated microneedles.

The microneedle can have a straight or tapered shaft. In one embodiment, the diameter of the microneedle is greatest at the base end of the microneedle and tapers to a point at the end distal the base. The microneedle can also be fabricated to have a shaft that includes both a straight (i.e., untapered) portion and a tapered portion. The microneedles can be formed with shafts that have a circular cross-section in the perpendicular, or the cross-section can be non-circular. The tip portion of the microneedles can have a variety of configurations. The tip of the microneedle can be symmetrical or asymmetrical about the longitudinal axis of the shaft. The tips may be beveled, tapered, squared-off, or rounded. The tip portion generally has a length that is less than 50% of the total length of the microneedle.

The dimensions of the microneedle, or array thereof, are designed for the particular way in which it is to be used. The length typically is selected taking into account both the portion that would be inserted into the biological tissue and the (base) portion that would remain uninserted. The cross-section, or width, is tailored to provide, among other things, the mechanical strength to remain intact for the delivery of the drug or for serving as a conduit for the withdrawal of biological fluid, while being inserted into the skin, while remaining in place during its functional period, and while being removed (unless designed to break off, dissolve, or otherwise not be removed). In various embodiments, the microneedle may have a length of between about 50 μm and about 5000 μm, preferably between about 200 μm and about 1500 μm, and more preferably between about 300 μm and about 1000 μm. In one embodiment, the length of the microneedle is about 750 μm. In various embodiments, the base portion of the microneedle has a width or cross-sectional dimension between about 20 μm and about 500 μm, preferably between about 50 μm and about 350 μm, more preferably between about 100 μm and 250 μm. For a hollow microneedle, the outer diameter or width may be between about 50 μm and about 400 μm, with an aperture diameter of between about 5 μm and about 100 μm. The microneedle may be fabricated to have an aspect ratio (width:length) between about 1:1.5 and 1:10. Other lengths, widths, and aspect ratios are envisioned.

In various embodiments, the microneedle device includes a single microneedle or an array of two or more microneedles. For example, the device may include an array of between 2 and 1000 (e.g., between 2 and 100) microneedles. In one embodiment, a device may include between 2 and 10 microneedles. An array of microneedles may include a mixture of different microneedles. For instance, an array may include microneedles having various lengths, base portion diameters, tip portion shapes, spacings between microneedles, drug coatings, etc.

The microneedle can be fabricated by a variety of methods known in the art or as described in the Examples below. Details of possible manufacturing techniques are described, for example, in U.S. Patent Application Publication No. 2006/0086689 A1 to Raju et al., U.S. Patent Application Publication No. 2006/0084942 to Kim et al., U.S. Patent Application Publication No. 2005/0209565 to Yuzhakov et al., U.S. Patent Application Publication No. 2002/0082543 A1 to Park et al., U.S. Pat. No. 6,334,856 to Allen et al., U.S. Pat. No. 6,611,707 to Prausnitz et al., U.S. Pat. No. 6,743,211 to Prausnitz et al., all of which are incorporated herein by reference.

In one embodiment, microneedles are fabricated on a flexible base substrate. It would be advantageous in some circumstances to have a base substrate that can bend to conform to the shape of the tissue surface. In another preferred embodiment, the microneedles are fabricated on curved base substrate. The curvature of the base substrate would be designed to conform to the shape of the tissue surface.

Drug can be coated onto the microneedle, integrated into the microneedle, passed through bores/apertures or channels in the microneedle, or a combination thereof.

### Drug Formulation

A wide range of drugs may be formulated for delivery to the eye with the present microneedle devices and methods. As used herein, the terms “drug” or “drug formulation” are used broadly to refer to any prophylactic, therapeutic, or diagnostic agent, or other substance that which may be suitable for introduction to biological tissues, including pharmaceutical excipients and substances for tattooing, cosmetics, and the like. The drug can be a substance having biological activity. The drug formulation may include various forms, such as liquid solutions, gels, solid particles (e.g., microparticles, nanoparticles), or combinations thereof. The drug may comprise small molecules, large (i.e., macro-) molecules, or a combination thereof. In representative, not non-limiting, embodiments, the drug can be selected from among amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, and viruses. The drug may be selected from suitable proteins, peptides and fragments thereof, which can be naturally occurring, synthesized or recombinantly produced. In one embodiment, the drug formulation includes insulin.

A variety of other pharmaceutical agents known in the art may be formulated for administration via the microneedle devices described herein. Examples include β-adrenoceptor antagonists (e.g., carteolol, cetamolol, betaxolol, levobunolol, metipranolol, timolol), miotics (e.g., pilocarpine, carbachol, physostigmine), sympathomimetics (e.g., adrenaline, dipivefrine), carbonic anhydrase inhibitors (e.g., acetazolamide, dorzolamide), prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), anti-viral compounds (e.g., acyclovir, cidofovir, idoxuridine, interferons), aldose reductase inhibitors, anti-inflammatory and/or anti-allergy compounds (e.g., steroidal compounds such as betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors.

The drug formulation may further include one or more pharmaceutically acceptable excipients, including pH modifiers, viscosity modifiers, diluents, etc., which are known in the art.

In preferred embodiments, the drug formulation can be tailored to flow through hollow microneedles from a reservoir. In alternative embodiments, the drug formulation may be coated onto the microneedle, can be included/integrated into the microneedle structure, or a combination thereof. The drug formulation may be coated onto microneedles by dip coating, spray coating, or other techniques known in the art.

### Control of Transport Through Microneedle

The transport of drug formulation or biological fluid through a hollow microneedle can be controlled or monitored using, for example, one or more valves, pumps, sensors, actuators, and microprocessors. For instance, the microneedle device may include a micropump, microvalve, and positioner, with a microprocessor programmed to control a pump or valve to control the rate of delivery of a drug formulation through the microneedle and into the biological tissue. The flow through a microneedle may be driven by diffusion, capillary action, a mechanical pump, electroosmosis, electrophoresis, convection, magnetic field, ultrasound, or other driving forces. Devices and microneedle designs can be tailored using known pumps and other devices to utilize these drivers. The microneedle devices can further include a flowmeter or other means to monitor flow through the microneedles and to coordinate use of the pumps and valves.

The flow of drug formulation or biological fluid can be regulated using various valves or gates known in the art. The valve may be one which can be selectively and repeatedly opened and closed, or it may be a single-use type, such as a fracturable barrier. Other valves or gates used in the microneedle devices can be activated thermally, electrochemically, mechanically, or magnetically to selectively initiate, modulate, or stop the flow of material through the microneedles. In one embodiment, the flow is controlled with a rate-limiting membrane acting as the valve.

### Uses of the Microneedle Device

The microneedle devices and operational techniques described herein may be used to deliver substances into and through the various biological tissues. In a preferred application, the microneedle devices and methods are used to deliver a drug, particularly a therapeutic, prophylactic, or diagnostic agent into the skin, sclera, or other biological tissue of a patient (i.e., a human, animal, or other living organism in need of therapeutic, diagnostic, or prophylactic intervention). In one embodiment, a hollow microneedle is inserted into the tissue, and then a region of the tissue is filled with a drug formulation by driving a fluid drug formulation through the microneedle, simultaneously with or following partial retraction of the microneedle from the tissue. The drug is then released from the region of the tissue over an extended period. The drug formulation may gel in the reservoir. The method provides sustained release of the drug over time.

In one embodiment, a hollow microneedle or array thereof is used to inject cells into biological tissue. This may be done for tissue engineering, or for stem cell or other cell-based therapies, which are described in the art.

In another embodiment, the drug formulation includes microparticles or nanoparticles of a drug, which optionally may be encapsulated, for example, with a biocompatible polymeric material. This may useful for sustained release of the drug following injection. The formulation preferably includes a fluid (e.g., a pharmaceutically acceptable carrier) in which the microparticles or nanoparticles may be suspended. Micro- and nano-encapsulation techniques are known in the art, as are aqueous and organic pharmaceutically acceptable carriers.

In one preferred embodiment, the microneedle device is used as a means to extract fluid out of a biological tissue. This fluid can subsequently be subjected to analysis to determine its physicochemical properties or the concentration or composition of one or more compounds present within the fluid. In one case, this fluid may be dermal interstitial fluid extracted from the skin, and the compound can be glucose, which is assayed for its concentration within the dermal interstitial fluid.

In another embodiment, the drug formulation is one which undergoes a phase change upon administration. For instance, a liquid drug formulation may be injected through hollow microneedles into the tissue, where it then gels within the tissue and the drug diffuses out from the gel to control release.

In an alternative embodiment, the microneedle itself is formed of a drug encapsulated polymer, which can be implanted into the biological tissue for drug release upon degradation/dissolution of the microneedle. The polymer may be a biodegradable one, such as a PLGA. In a specific case, the microneedle or the tip portion thereof is intentionally broken off in the tissue, and will biodegrade and release drug. In still another embodiment, the microneedle is formed of metal and coated with a drug formulation, which delivers the drug by dissolution/diffusion upon insertion into the tissue. In a preferred embodiment, the drug coating is water soluble.

The microneedle devices also may be adapted to use the one or more microneedles as a sensor to detect analytes, electrical activity, and optical or other signals. The sensor may include sensors of pressure, temperature, chemicals, and/or electromagnetic fields (e.g., light). Biosensors can be located on the microneedle surface, inside a hollow or porous microneedle, or inside a device in communication with the body tissue via the microneedle (solid, hollow, or porous). The microneedle biosensor can be any of the four classes of principal transducers: potentiometric, amperometric, optical, and physiochemical. In one embodiment, a hollow microneedle is filled with a substance, such as a gel, that has a sensing functionality associated with it. In an application for sensing based on binding to a substrate or reaction mediated by an enzyme, the substrate or enzyme can be immobilized in the needle interior. In another embodiment, a wave guide can be incorporated into the microneedle device to direct light to a specific location, or for detection, for example, using means such as a pH dye for color evaluation. Similarly, heat, electricity, light or other energy forms may be precisely transmitted to directly stimulate, damage, or heal a specific tissue or for diagnostic purposes.

The present invention may be further understood with reference to the following non-limiting examples.

### EXAMPLE 1

### Fluid Delivery Through Hollow Microneedles Into Human Cadaver Skin

### Effect of Partial Retraction Following Insertion

Single microneedles were inserted into human cadaver skin in vitro to a controlled depth and then, sometimes, partially retracted.

### Microneedle Fabrication

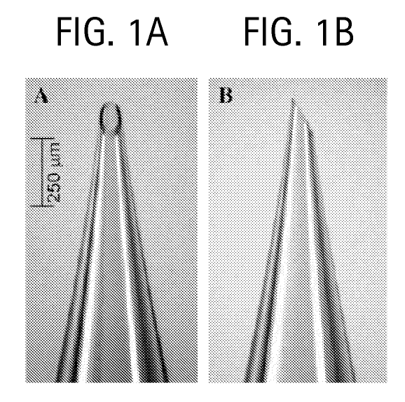

Glass microneedles were fabricated by pulling fire-polished borosilicate glass pipettes (o.d. 1.5 mm, i.d. 0.86 mm, BF150-86-15, Suffer Instrument, Novato, Calif.) using a micropipette puller (P-97, Sutter Instrument). In most cases, the resulting blunt-tip microneedles were then beveled (BV-10, Sutter Instrument) and cleaned using chromic acid (Mallinckrodt, Hazelwood, Mo.), followed by filtered DI water and acetone (J. T. Baker, Phillipsburg, N.J.) rinses. Microneedle geometries were determined by bright-field microscopy (Leica DC 300; Leica Microsystems, Bannockburn, Ill.) and image analysis (Image Pro Plus, version 4.5, Media Cybernetics, Silver Spring, Md.). The microfabricated microneedles typically had an effective tip opening radius of 22 to 48 μm with a tip bevel angle of 35 to 38°. Because the opening of a bevel-tip microneedle was oval in shape, the effective radius was determined as the average of the half-lengths of the long and short axes of the ellipse. FIG. 1 shows a representative glass microneedle as used in these experiments.

### Skin Preparation

Human abdominal skin was obtained from cadavers and stored at −80° C. (Revco Ultima II, Kendro Laboratory Products, Asheville, N.C.). After warming to room temperature and removing subcutaneous fat, skin was hydrated in a Pyrex dish filled with phosphate-buffered saline (PBS; Sigma, St. Louis, Mo.) for at least 15 min prior to use. The skin was then cut into 4 cm×4 cm pieces and stretched onto a stainless steel specimen board with eight tissue-mounting pins on it to mimic the tension of living human skin.

### Infusion Experiments

To measure flow rate into skin during microneedle infusion, a single microneedle was inserted into human cadaver skin to microinfuse sulforhodamine solution and the infusion flow rate was measured over time. As an aid to visualizing flow into skin, sulforhodamine-B dye (Molecular Probes, Eugene, Oreg.) was added to PBS, stirred, and filtered (0.2 μm pore size, Nalge Nunc International, Rochester, N.Y.) to make 1×10⁻³ M sulforhodamine solution. Either a 250 μl or 1 ml glass syringe (Gastight Syringe, Hamilton Company, Reno, Nev.) was used as the reservoir for sulforhodamine solution and connected to a high-pressure CO₂ gas tank (Airgas, Radnor, Pa.) on one end and connected to a 2.1 mm i.d. metal tubing line on the other end, which was then connected to the end of a microneedle using a short, flexible tubing linker. A custom-made, rotary-threaded device (see Wang, et al., Diabetes Technol Ther 7:131-41 (2005)) was used to hold the microneedle and allow microneedle insertion into and retraction out of the skin in a controlled fashion calibrated by the number of rotations of the device (i.e., 1 full rotation=1440 μm needle displacement). This assembly was held by a stainless steel adapter attached to a Z-stage (Graduated Knob Unislide, Velmex, Bloomfield, N.Y.) to control vertical motion of the microneedle holder with ±10 μm resolution.

The flow rate of sulforhodamine solution microinjection was determined by following movement of the gas-liquid meniscus in the syringe reservoir over time with a digital video camera (DCR-TRV460, Sony, Tokyo, Japan) and image analysis software (Adobe Photoshop 7.0, Adobe Systems, San Jose, Calif.) after converting the captured movie into sequences of still images (Adobe Premiere 6.0, Adobe Systems). After initially positioning the gas-liquid meniscus at the top of the glass syringe by injecting an appropriate amount of air upstream from the syringe, the microneedle was lowered until its tip touched the skin sample. The microneedle holder was then rotated clockwise to insert the microneedle to the desired insertion depth and sometimes rotated counterclockwise to retract the microneedle to the desired retraction distance.

The gas pressure was then set to the desired infusion pressure using a pressure regulator (Two-Stage Regulator, Fisher Scientific, Hampton, N.H.) on the CO₂ cylinder and the experiment began. During the process, the skin was examined for fluid leakage, which was easily visible due to the presence of sulforhodamine dye in the fluid. If no leakage was observed, then the flow rate within the glass syringe was assumed to be equal to the flow rate into the skin. This assumption was validated by quantifying the sulforhodamine content of skin after microinjection using spectrofluorimetry after chemically digesting the skin. Data were discarded in those few cases (<5%) where leakage was observed.

To serve as a base case experiment, a bevel-tip microneedle with 30-μm effective opening radius was inserted into the skin to a depth of 1080 μm and microinfusion flow was initiated by applying a constant infusion pressure of 138 kPa.

Every 5 min, the needle was retracted 180 μm back toward the skin surface to a final insertion depth of 180 μm (relative to its initial position prior to insertion). The infusion flow rate was measured every minute with a 5 s offset at the beginning and the end of each period at a given retraction position (i.e., at 5 s, 60 s, 120 s, 180 s, 240 s, and 295 s).

To determine the effects of infusion parameters, microneedle infusion flow rates were measured over a range of different experimental conditions. To determine the effect of microneedle insertion depth and retraction distance on flow rate, the base case experiment was compared to otherwise identical experiments using 900-μm and 720-μm initial insertion depths followed by 180-μm retraction every 5 min. The effect of infusion pressure was determined by comparing to pressures of 69 and 172 kPa. The effect of tip bevel was determined by comparing to a blunt-tip microneedle with the same opening (i.e., 30 μm radius) as the bevel-tip microneedle. The effect of microneedle tip opening size was determined by comparing to needles with smaller (22 μm radius) and larger (48 μm radius) tip openings. Finally, the effect of infusion time was determined over longer times by inserting the microneedle to a depth of 1080 μm and either leaving it in place or retracting 720 μm to a final insertion depth of 360 μm and measuring flow rate for 104 min.

### Histological and Microscopic Image Analysis

After each infusion experiment, the top surfaces and undersides of the skin were imaged using bright-field microscopy (Leica DC 300) to visualize the distribution of sulforhodamine infusion. Additional experiments were performed for detailed histological imaging of microneedle penetration pathways and injection within skin. For these experiments, a microneedle with 30-μm effective radius opening and 38° bevel angle was externally spray-coated (Testor Corporation, Rockford, Ill.) using a solution of red dye (Tissue Marking Dye; Triangle Biomedical Sciences, Durham, N.C.) thickened using 2×10⁻² M polyvinylpyrrolidone (Aldrich, St. Louis, Mo.). The needle was then inserted to a depth of 1080 μm into the skin and retracted 720 μm to a final insertion depth of 360 μm. A solution containing 2.1×10⁸ particles/ml of 6-μm yellow-green fluorescence-labeled polystyrene microspheres (Fluoresbrite™ YG, Polysciences, Warrington, Pa.) mixed with blue-green dye (Triangle Biomedical Sciences) was injected at 138 kPa infusion pressure until the dye became visible within the skin (˜1 min infusion time). These skin samples, with microneedles in place, were fixed using 10% neutral buffered formal in (Fisher Scientific) or a mixture of 2% paraformaldehyde and 2% glutaraldehyde (Electron Microscopy Sciences, Hatfield, Pa.) for at least 24 h before rinsing thoroughly with PBS and then submerging in sterilized, filtered, 30% sucrose overnight (see Bancroft & Gamble, Theory and Practice of Histological Techniques, Churchill Livingstone, New York, N.Y. 2002). The skin samples were frozen in liquid nitrogen with optimal cutting temperature compound (Tissue-Tek, Sakura Finetek, Torrance, Calif.) in an embedding mold container and later sectioned into 10-μm thick slices using a cryostat (Cryo-star HM 560MV, Microm, Waldorf, Germany). Histological sections were surface stained with hematoxylin and eosin (Autostainer XL, Leica Microsystems) and examined by bright field (Leica DC 300) and fluorescence microscopy (Eclipse E6000W, Nikon, Melville, N.Y.).

### Statistical Analysis

Flow rate measurements at each condition and time point were performed using at least three skin specimens, from which the mean and standard deviation were calculated. A two-tailed Student's t-test (α=0.05) was performed when comparing two experimental conditions. When comparing three or more experimental conditions, a one-way analysis of variance (ANOVA α=0.05) was performed. A two-way analysis of variance (ANOVA α=0.05) was performed when comparing two factors. In all cases, a value p<0.05 was considered statistically significant.

### Results

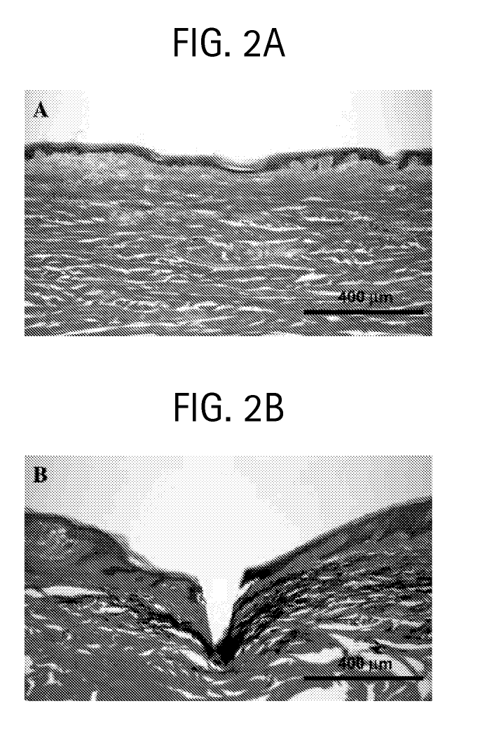

FIG. 2 shows a cross section of a piece of skin pierced with a microneedle and then chemically fixed for histological sectioning and staining. The hole made by the microneedle is evident, measuring 300 to 350 μm deep and having the same shape as the microneedle. Some deformation of the skin surface is also evident, due to skin deflection during insertion. A small volume of dye was injected into the skin and its infusion trajectory is also shown in FIG. 2. More dye is present on the right side of the image, presumably because the needle bevel was on the right and channeled flow in that direction. The directional nature of the flow further appears to be influenced by skin microstructure, where infusion pathways follow dermal collagen fiber orientation.

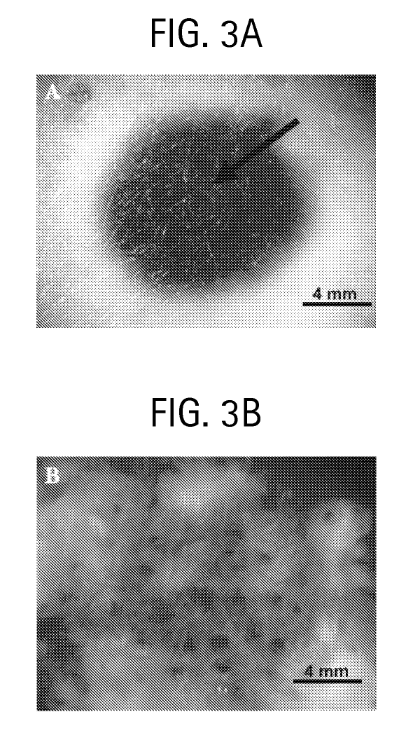

In a typical experiment, hundreds of microliters of fluid were infused into skin, which was distributed over an area measuring millimeters to centimeters across. Infusion of 755 μl of solution was carried out over 104 min at 138 kPa using a microneedle with a 31 μm effective radius opening and a bevel angle of 37° inserted to a depth of 1080 μm into the skin and retracted 720 μm to a final insertion depth of 360 μm. FIGS. 3A-B shows representative images of the top and bottom surfaces of human cadaver skin after sulforhodamine infusion using a microneedle. Sites of sulforhodamine infusion are indicated by dark red staining. All dye was contained within the skin and did not represent surface staining; leakage onto the skin surface during infusion was rarely seen. Dye distribution observed from the skin surface, as shown as the circular dark area in FIG. 3A, was generally more constrained than when viewed from underneath the skin, as shown by the widespread staining in FIG. 3B. Additional studies showed that microneedles could also be inserted into pig and rat cadaver skin to microinfuse sulforhodamine solution in a similar manner.

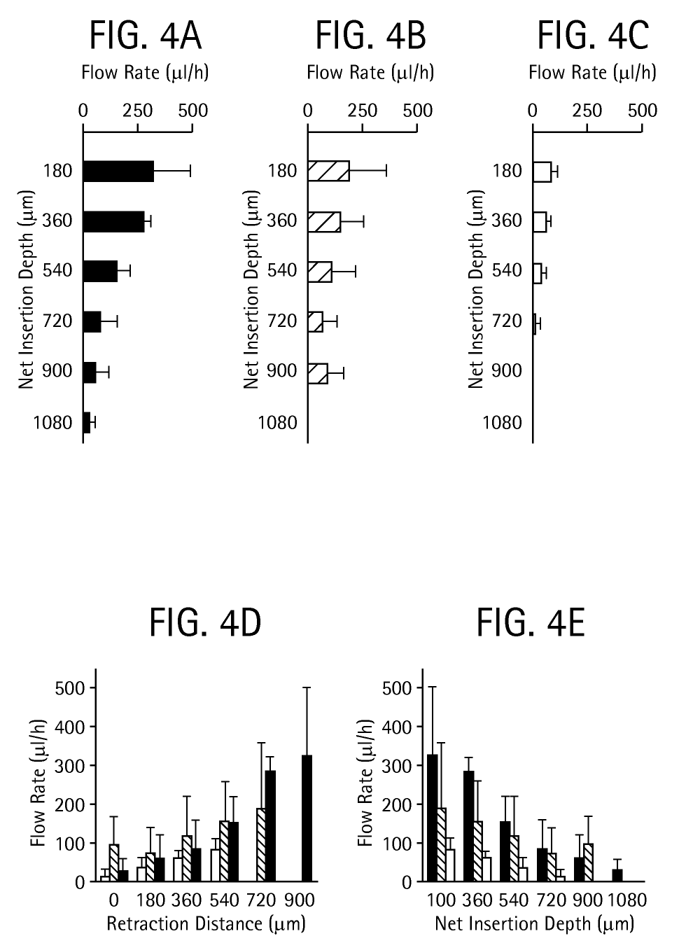

Microneedles were initially inserted to a maximum insertion depth of (A) 1080 μm, (B) 900 μm or (C) 720 μm and then retracted various distances back toward the skin surface to a final, net insertion depth. Microneedles had tip opening effective radii of 27-31 μm with bevel angles of 35-37°. Infusion was performed for 5 min at 138 kPa without needle retraction, after which, microneedles were retracted by 180 μm every 5 min to a final insertion depth of 180 μm. Microneedle insertion to a depth of 1080 μm without retraction permitted sulforhodamine solution flow into the skin at a rate of 28 μl/h (FIG. 4A). Retraction of the microneedles led to progressively larger flow rates, where a 900 μm retraction, corresponding to a net insertion of 180 μm, produced a flow rate of 326 μl/h. Initial insertion to lesser depths similarly produced low flow rates that were progressively increased with needle retraction (FIG. 4B and FIG. 4C). When these data from different insertion and retraction combinations are combined, a significant increase in flow rate with increasing retraction distance is apparent (FIG. 4D; ANOVA, p<0.05). Further analysis also showed a significant increase in flow rate with increasing initial insertion depth (ANOVA; p<0.001). Decreasing the net insertion depth, which corresponds to the initial insertion depth minus the retraction distance, also resulted in a larger flow rate (FIG. 4E; ANOVA, p<0.05). Pooling the data from parts (A), (B), and (C), flow rate is shown as a function of (D) retraction distance and (E) net insertion depth for maximum insertion depths of 720 μm (white bars), 900 μm (striped bars) and 1080 μm (black bars). Data are expressed as mean values (n≧3) with standard deviation bars. Considered together, these findings demonstrate that flow rate is largest when retraction distance is maximized by inserting deeply and retracting almost completely.

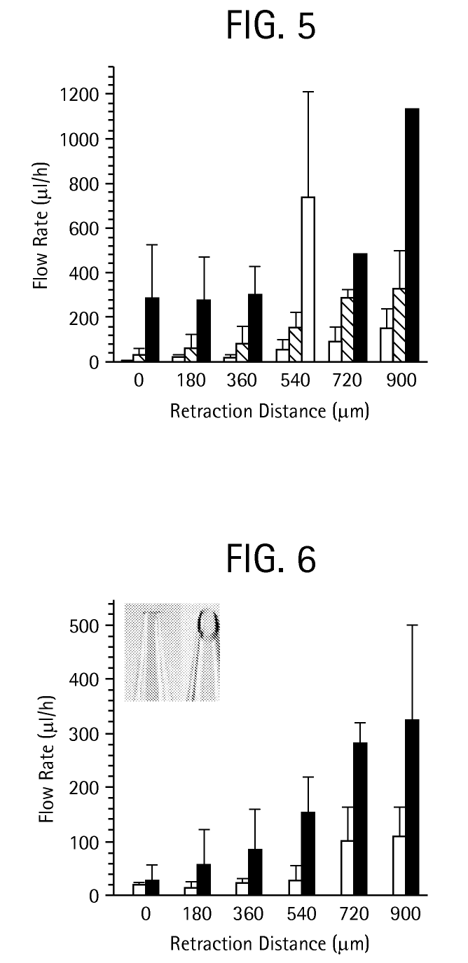

Infusion pressure also affects flow rate. As shown in FIG. 5, flow rate increased with increasing pressure (ANOVA, p<0.0001). Microinfusion flow rate was measured as a function of retraction distance at three pressures: 69 kPa (white bars), 138 kPa (striped bars) and 172 kPa (black bars). Although an increased infusion pressure increased flow rate by increasing the pressure-driven driving force for flow, it might also act by displacing or deforming tissue to reduce resistance to flow. When separating these two effects, it is expected that flow rate should increase in direct proportion to pressure if pressure acts only as a driving force for convection (Fox & McDonald. Introduction to Fluid Mechanics, Wiley, New York, N.Y., 1998). In contrast, pressure-mediated changes in skin microstructure that reduce flow resistance are expected to have a nonlinear dependence on pressure (Zhang, et al., Am J Physiol Heart Circ Physiol 279:H2726-34 (2000); McGuire & Yuan, Am J Physiol Heart Circ Physiol 281:H715-21 (2001)). The flow rate data shown in FIG. 5 were normalized by the infusion pressure. Data are expressed as mean values (n≧3) with standard deviation bars. From 69 to 138 kPa, the increase in flow rate was proportional to the increase in pressure (ANOVA, p=0.14). However, at 172 kPa, the flow rate showed a disproportionately large increase relative to the pressure increase (ANOVA, p<0.001). This nonlinearity suggests that elevated pressure reduced flow resistance by displacing or deforming the dense dermal tissue impeding flow.

Dermal tissue compressed by needle insertion should be most dense at the needle tip. Thus, flow from a blunt-tip microneedle, where the hole is located at the needle tip, should encounter greater resistance than flow from a bevel-tip microneedle, where the hole is off to the side. Consistent with this hypothesis, infusion flow rate from a bevel-tip microneedle was on average 3-fold greater than from a blunt-tip microneedle (FIG. 6; ANOVA, p<0.0001). Microinfusion flow rate was measured as a function of retraction distance during infusion using hollow microneedles with a blunt tip (white bars; left inset image) and a 35° beveled tip (black bars; right inset image).

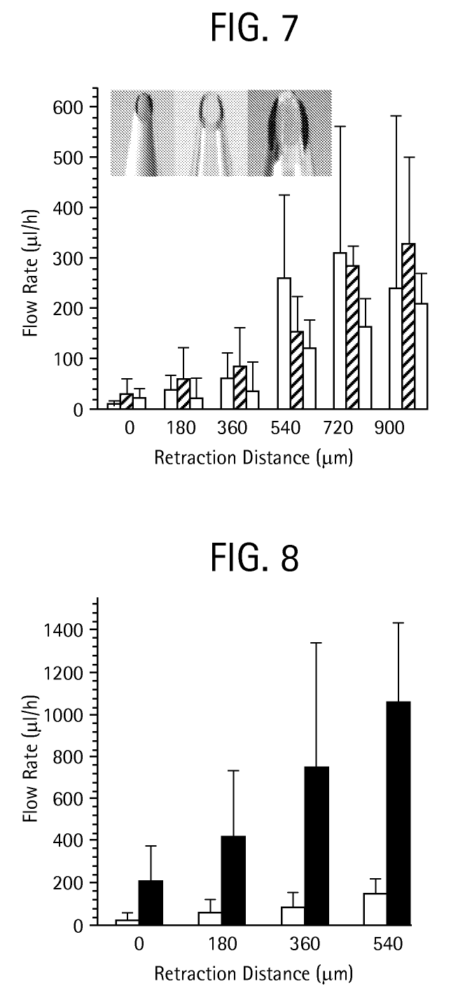

If dense dermal tissue is the main barrier to flow, the size of the microneedle tip opening is expected to have only small effects since the microneedle itself does not impose the rate-limiting barrier. Consistent with this expectation, varying tip openings with effective radii ranging from 22 to 48 μm (i.e., tip areas ranging from 1462 to 7400 μm²) had no significant effect on infusion flow rate (FIG. 7; ANOVA, p=0.27). Microinfusion flow rate was measured as a function of retraction distance using three different tip opening sizes: 22 μm (black bars; left inset image), 30 μm (striped bars; center inset image) and 48 μm (white bars; right inset image) effective radii. Data are expressed as mean values (n≧3) with standard deviation bars. This suggests that the primary resistance to flow is not at the needle tip-to-skin interface, which would be heavily influenced by a 5-fold change in area. Instead, the volume of dense dermal tissue surrounding the tip appears to limit flow, where the size and density of this tissue is probably governed primarily by the depth of needle insertion and retraction, with little influence from tip opening size.

### EXAMPLE 2

### Effect of Hyaluronidase on Microinfusion Flow

Hyaluronidase is known to reduce flow resistance in the skin by rapidly breaking down hyaluronan, a glycosaminoglycan within skin collagen fibers (Kreil, Protein Sci 4:1666-69 (1995); Bruera, et al., Annals of Oncology 10:1255-58 (1999); Meyer, “Hyaluronidases” in The Enzymes, vol. 5, pp. 307-20 (Boyer, ed) Academic Press, New York, N.Y. (1971)). This enzyme might similarly break down the resistance of dermal tissue compressed during microneedle insertion. To test this prediction, microneedle infusion was carried out using the sulforhodamine solution described in Example 1 mixed with a purified ovine testicular hyaluronidase preparation (Vitrase™) that is commercially available and FDA approved for human use to facilitate injection when simultaneously co-injected using a hypodermic needle (Hyaluronidase (Vitrase)—ISTA. Drugs in R&D 4:194-97 (2003)).

The effect of hyaluronidase was determined by comparing to an infusion fluid containing 200 U/ml hyaluronidase prepared by mixing 3 ml of hyaluronidase solution (Vitrase™, 200 U/ml, ISTA Pharmaceuticals, Irvine, Calif.) with 7 mL of 1×10⁻³ M sulforhodamine solution. Testing was conducted as described in Example 1. Microneedles having 35-37° beveled tips with 27-32 μm effective radius openings were inserted to a maximum depth of 1080 μm and infusion was carried out at 138 kPa. Addition of hyaluronidase increased infusion flow rate by 7 fold (FIG. 8; ANOVA, p≦0.01). Microinfusion flow rate was measured as a function of retraction distance during infusion in the absence (white bars) or presence (black bars) of hyaluronidase. Data are expressed as mean values (n≧3) with standard deviation bars. This finding further supports the global hypothesis that dense dermal tissue limits flow from microneedles. It also suggests applications, where hyaluronidase or other compounds that affect skin microstructure could be co-administered to facilitate infusion using microneedles.

### EXAMPLE 3

### Tissue Resistance to Microinfusion Flow

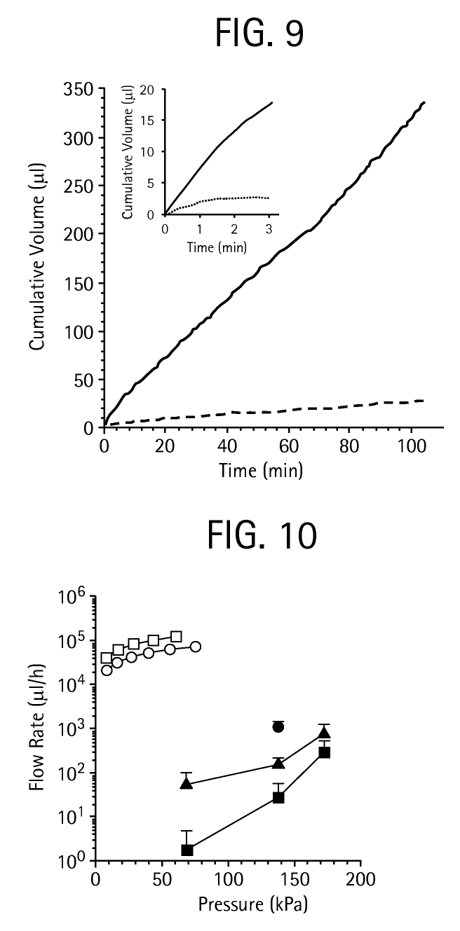

The resistance to fluid flow into tumor tissue at constant pressure has been shown to decrease over time, probably due to flow-induced changes in tissue microstructure. To address this possibility during infusion using microneedles as described in Example 1 above, the flow into skin was measured continuously for 100 min for needles inserted and left in place and for needles inserted and retracted (FIG. 9). The microneedles were inserted into skin to a depth of 1080 μm and then retracted 720 μm to a final insertion depth of 360 μm (solid line) and microneedles inserted to a depth of 1080 μm without retraction (dashed line). Infusion was carried out at 138 kPa using microneedles having 35-38° beveled tips with 30-32 μm effective radius openings. Data are expressed as mean values (n≧3) with average standard deviation of 40% for both curves (not shown). In both cases, the cumulative volume of fluid infused into skin increased linearly with time (linear regression correlation coefficient, R²>0.99), suggesting that skin resistance to flow did not change over the timescale studied. The pressure used in this experiment was less than the pressure identified previously to induce non-linear changes in flow rate (FIG. 5), which is consistent with this observation that flow rate did not change with time. Despite the generally linear trend, the flow rate was transiently higher at the onset of flow and then decayed within a few minutes to a steady state (FIG. 9 inset). This effect might be explained by the rapid filling of a small cavity in the skin at the needle tip formed by the insertion/retraction process, followed by slower infusion into and across intact, compressed tissue.

Tissue resistance is expected to be the dominant resistance to flow when compared to the resistance offered by the microneedle itself during a microneedle injection into skin. To test this hypothesis, flow rate measurement from a previous study (Martanto, et al., AIChE J 51:1599-607 (2005)), which corresponds to flow of fluid through microneedles into the air (i.e., without the presence of skin for microneedles with radii of 25 and 34 μm), was plotted together with a subset of flow rate measurements in FIG. 5 and FIG. 8, which correspond to flow of fluid through microneedles during microinfusion into skin (i.e., at infusion pressures of 69, 138, 172 kPa with and without the presence of hyaluronidase). This comparison is shown in FIG. 10, which indicates that the presence of skin tissue decreases the flow rate by up to a few orders of magnitude. Fluid flow rate was measured as a function of pressure across microneedles with (filled symbols) and without (empty symbols) the presence of skin. Fluid flow rates through microneedles with (∘) 25 μm and (□) 34 μm radii were compared to those with the skin present, in which microinfusion into skin using microneedles was performed with insertion depth of 1080 μm without retraction (▪), with insertion depth of 1080 μm and retracted 540 μm without the presence of hyaluronidase (▴) and with the presence of hyaluronidase () (data from FIG. 5 and FIG. 8). Data are expressed as mean values (n≧3) with standard deviation bars. Overall this result suggests that dermal tissue offers significant resistance to flow during microinfusion using microneedles into skin.

### EXAMPLE 4

### Controlled Insertion for Targeted Fluid Delivery Through Hollow Microneedles In Vivo and In Vitro

### Microneedle Fabrication

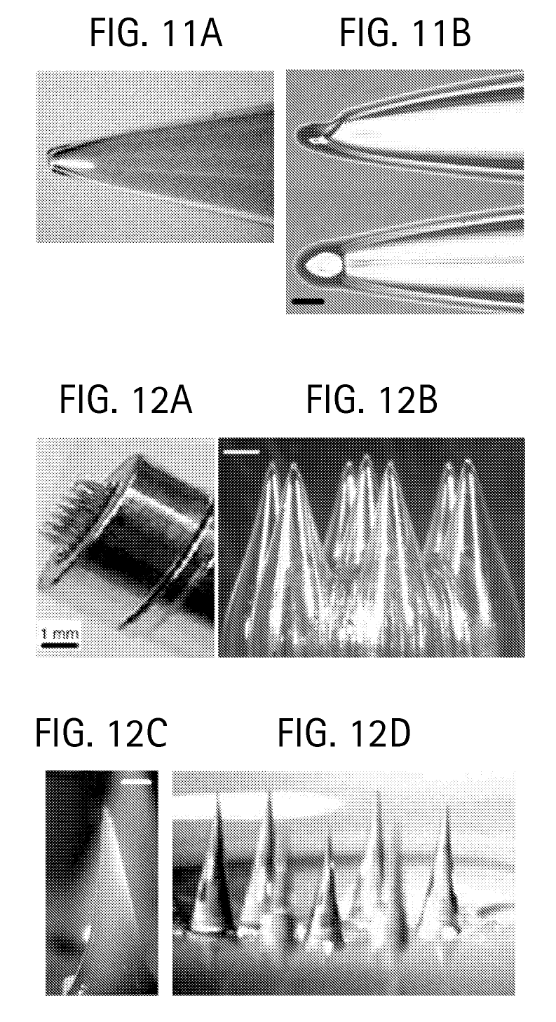

Glass microneedles were made using conventional drawn-glass micropipette techniques as described in Example 1. The blunt glass microneedles were fabricated with outer radii at the tip of 15-40 μm and cone angles of 20-30° (FIG. 11A). The resulting blunt-tip microneedles were then beveled as described in Example 1 to produce a beveled tip with a side-opening bore having an oval geometry with a short ellipsoidal axis of 60-110 μm and a long ellipsoidal axis of 80-160 μm (FIG. 11B). The microneedles were cleaned as described in Example 1. To increase their strength, the microneedle tips were quickly heated using a Bunsen burner for 1-2 s to smooth and thicken the needle wall at the tip. Some of the microneedles were assembled into seven-needle bundles using epoxy resin (Polypoxy 1010, Polytek, Easton, Pa.) to form multi-needle arrays (FIGS. 12A-B).

Polymer microneedles were fabricated by first making a mold out of polydimethylsiloxane (Sylgard 184, Dow Corning, Midland, Mich.) from a master array of glass microneedles and then using the mold to make replicate arrays of polymer microneedles by casting with poly-glycolide or poly-lactide-co-glycolide (Absorbable Polymers International, Pelham, Ala.) (Park et al., J Control Release 104:51-66 (2005)) (FIG. 12C,D). Microneedle geometry was imaged and measured by microscopy.

### Skin Preparation

The in vitro skin model was full-thickness human cadaver skin. Skin samples were stored at −70° C. and thawed to room temperature before use. The in vivo skin model was healthy, anesthetized, adult, male, Sprague-Dawley hairless rats. Experimental skin specimens were harvested post mortem for histological analysis.

### Infusion Experiments

Single, hollow, glass microneedles were inserted into the back skin of anesthetized rats placed in a prone position or into human cadaver skin (2×2 cm²) placed on a x-y stage. Compounds delivered included blue tissue-marking dye in PBS (20% v/v, Shandon, Pittsburg, Pa.), green-fluorescent calcein in PBS (10 μM-10 mM, Molecular Probes, Eugene, Oreg.).

Microneedles were first filled from the distal end with the above solutions or suspensions using a conventional hypodermic needle (25-31 gauge, Becton Dickinson, Franklin Lakes, N.J.); a thin filament manufactured inside the microneedle lumen enhanced liquid flow to the proximal/tip end of the microneedle and displace air bubbles. Microneedles were connected via tubing to a cylinder of compressed CO₂ which drove microinfusion at regulated pressures of 5-20 psi (gauge) for delivery durations of 5-30 min.

Microneedles were mounted in a rotary drilling device, which controlled axial translation of the microneedle tip with ±10 μm accuracy during drilling into the skin. Device calibration by microscopy determined that every 360° rotation of the drilling device moved the microneedle tip 1,200 μm in its axial direction. However, due to skin deformation during insertion, microneedle insertion depth into the skin was only ˜800 μm, as determined by subsequent histological examination of skin samples. The amount of this deformation was limited not only by the drilling method of insertion, but also by pressing the convex based of the insertion device firmly against the skin surface, which held the skin in place.

### Histological and Microscopic Analysis

After microinfusion experiments, skin samples were preserved as described in Example 1. Histological sections of skin were examined by stereoscopic (SZX12, Olympus, Melville, N.Y.) and fluorescence microscopy (IX 70, Olympus). Images were recorded by a digital camera (Spot RT Slider camera, Model 231, Diagnostic Instruments, Sterling Heights, Mich.) and analyzed using image analysis software (Image Pro Plus, version 4.5, Media Cybernetics, Silver Spring, Md.). The volume of injected liquid was imaged in the skin for quantitative analysis using one of three different methods.

The first method analyzed sectioned skin slices by digital microscopy to determine the area stained by microinjection of a blue-green tissue marking dye solution. Multiplying this area by the skin section thickness provided a volume. Summing the volumes from all skin sections containing dye yielded the total injected volume.

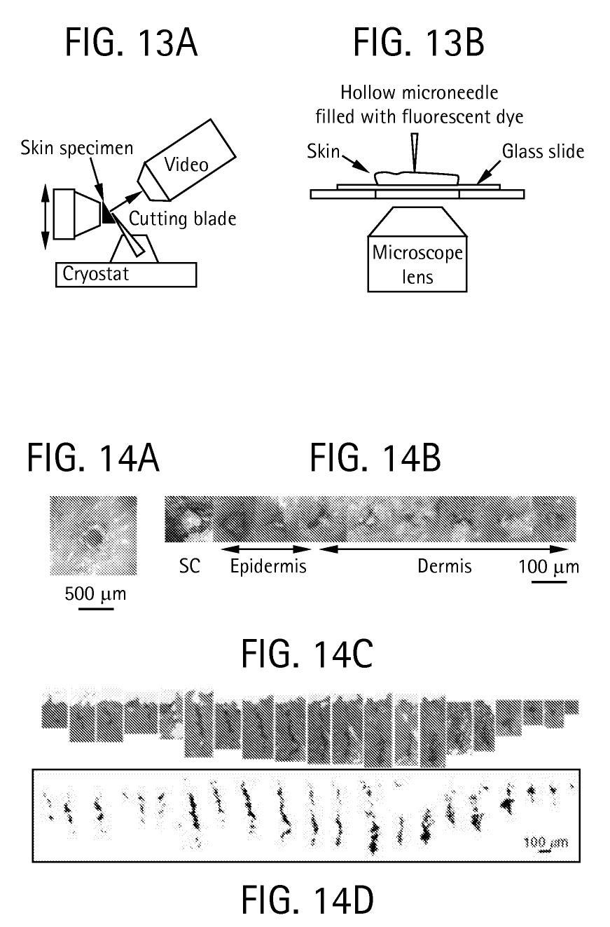

The second method used a mini-video camera (with a resolution of 640×480 pixels), positioned at the microtome stage oriented perpendicularly to the sectioning surface of the skin specimen (FIG. 13A) to directly image each sectioned skin slice on the microtome. The images were stored in a computer for further analysis and the skin sections were discarded. The total injected volume was calculated as described above, based on the dyed area and skin section thickness.

The third method measured microinfusion into skin in situ in real time. A microneedle filled with fluorescent dye solution of calcein was rotary drilled 500-1000 μm into a piece of skin (1 cm²) placed with the stratum corneum side facing up on a glass slide on the stage of an inverted microscope (FIG. 13B). The microscope was focused on the tip of the microneedle and a time series of images was collected every 2 s while infusing calcein solution at a pressure of 10 psi. The relative infusion rate over time was determined by assuming that fluorescence measured in the images was proportional to the injected volume. The injected volume on an absolute basis was determined by measuring the change in weight of the liquid-filled microneedle before and after the injection assuming a liquid density of 1 g/ml using a balance with a sensitivity of ±0.1 mg.

### Statistical Analysis

All data are presented as mean±standard error of the mean. Student's t-test was used to assess statistically significant differences. Comparisons between group means were carried out using analysis of variance (ANOVA). Mean values were considered significantly different if P<0.05. An unpaired t-test was used to determine the significance of the differences of blood glucose levels caused by microinjection of insulin and saline. All statistical analyses were performed using Statview (SAS Institute, Cary, N.C.).

### Results

A hand-operated, rotary penetration device was developed to insert microneedles into the skin of hairless rats in vivo and human cadaver skin in vitro in a controlled manner by drilling individual microneedles into the skin to a predetermined depth regulated by the number of turns of the device. This rotary drilling approach was designed to reduce the force of insertion into skin, based on the expectation that drilling insertion should cause less skin deformation than direct penetration. After insertion in this manner, the glass microneedles were found to be mechanically robust; needles could be inserted into the skin repeatedly (i.e., >10 times) without damage to the needle, as determined by microscopy.

The hole punctured in the skin by microneedles could be viewed after inserting and removing the microneedle. When viewed in situ from the skin surface of a hairless rat, holes with diameters ranging from 100 to 200 μm were imaged by microscopy (e.g., FIG. 14A). These holes on the surface on hairless rat skin disappeared within 10-20 min in vivo, based on visual observation through a stereomicroscope. Only mild, localized erythema was observed immediately after microneedle treatment, which disappeared within minutes. These kinetics are similar to observations made in human subjects (Kaushik et al. 2001). Microneedle insertion and injection caused no significant complications in the animals.

Histological sectioning of skin in vitro shows the shape and depth of needle penetration pathways. FIG. 14B shows a series of sequential cross-sectional images taken parallel to the skin surface, which gives an indication of the pathway geometry at different depths in the skin. FIG. 14C shows a series of sequential cross-sectional images taken perpendicular to the skin surface and stained with tissue-marking dye injected by the microneedle. These images show a tapered pathway with a geometry similar to that of the microneedle.

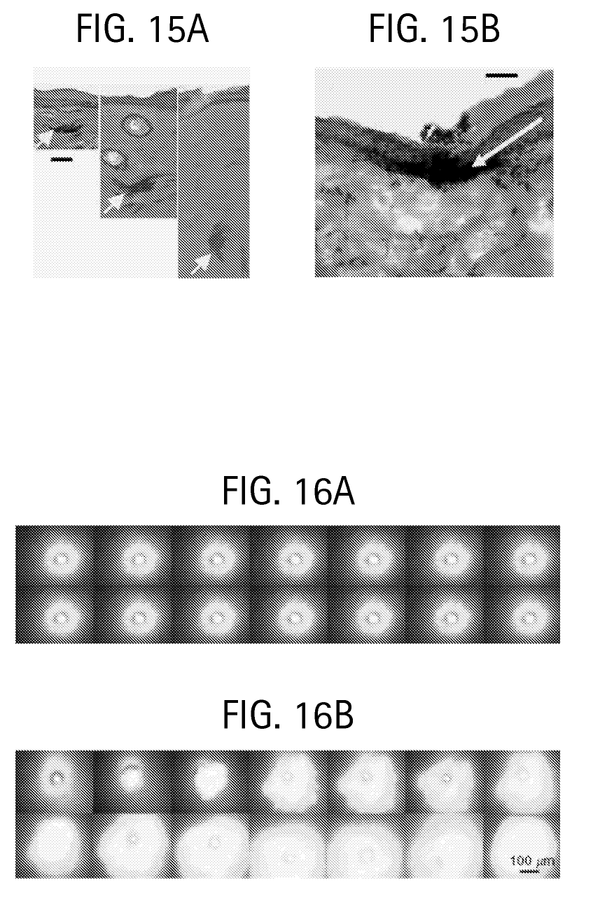

The rotary drilling method was designed both to reduce the force required for penetration into skin and to facilitate microinjection at precise locations within the skin. To quantify the precision and reproducibility of targeted insertion, microneedles were inserted into the back skin on hairless rats in vivo at six predetermined depths over the range 150-770 μm deep (corresponding to 180-1080° rotation of the insertion device). FIG. 15A demonstrates that injections were made at three different predetermined depths within the dermis of hairless rats in vivo. In this case, injection depth ranged from just below the dermal-epidermal junction to the mid-dermis. Injection in this region may be of interest to target delivery to the rich capillary bed found in the superficial dermis for uptake and systemic distribution in the bloodstream. As shown in FIG. 15B, even shallower skin penetration and injection can target delivery to the epidermis, which may be useful for various dermatological therapies, as well as for vaccine delivery to Langerhans dendritic cells found in the epidermis.

Based on three replicates at each condition, the average standard error was ±60 μm on an absolute basis and ±16% on a percent basis. The exact relationship between rotation of the insertion device and depth of penetration into the skin is a complex function of skin mechanical properties, needle geometry, and other factors. The microneedles were inserted to desired depths under the controlled and reproducible conditions used in this study. Controlled insertion at different skin sites, using different needle geometries, or other possible variations can be ascertained using routine experimentation.

The process of microinjection was imaged in situ by piercing hairless rat cadaver skin from above with a microneedle and viewing flow of fluorescent solution into the skin from below in real time. Insertion of a microneedle in this manner showed local injection of fluorescent solution, consistent with imaging results above, but flow into skin was slow (FIG. 16A). Slightly retracting the needle had a large effect on flow into skin (FIG. 16B). Within a few seconds, the field of view imaged by the microscope was filled with fluorescent solution. Macroscopic examination of the tissue afterwards showed fluorescent solution infusion over much larger areas, depending on the duration of microinfusion. Images were recorded every 2 s using real-time microscopy. In FIG. 16A, the microneedle was inserted to a depth of approximately 800 μm and flow was initiated at the first frame. No significant change in fluorescence was observed, corresponding to little or no injection into the skin. In FIG. 16B, the microneedle was initially inserted to the same depth, but was then retracted approximately 200 μm before initiating flow at the first frame. Large increases in fluorescence was observed, corresponding to rapid infusion into the skin. Absolute quantification of flow rates in this case was difficult; however, measurements carried out in human cadaver skin using a similar apparatus in a separate study measured flow rates of 21-1,130 μl/h through individual microneedles, which increased strongly with microneedle retraction (Martanto et al., Pharm Res 23:104-13 (2006)).

### EXAMPLE 5

### Delivery of Insulin by Microinjection

To demonstrate delivery of a bioactive macromolecule, insulin solution was microinjected into hairless rat skin in vitro and in vivo using diabetic rat models as described previously (see Martano, et al., Pharm Res 21:947-52 (2004)). During insulin delivery experiments, rats were anesthetized. Microneedle infusion was carried out as described in Example 4, using clinical insulin solution (100 U/ml, Humulin R, Eli Lilly, Indianapolis, Ind.), and FITC-labeled insulin in PBS (1 mg/ml, Sigma).

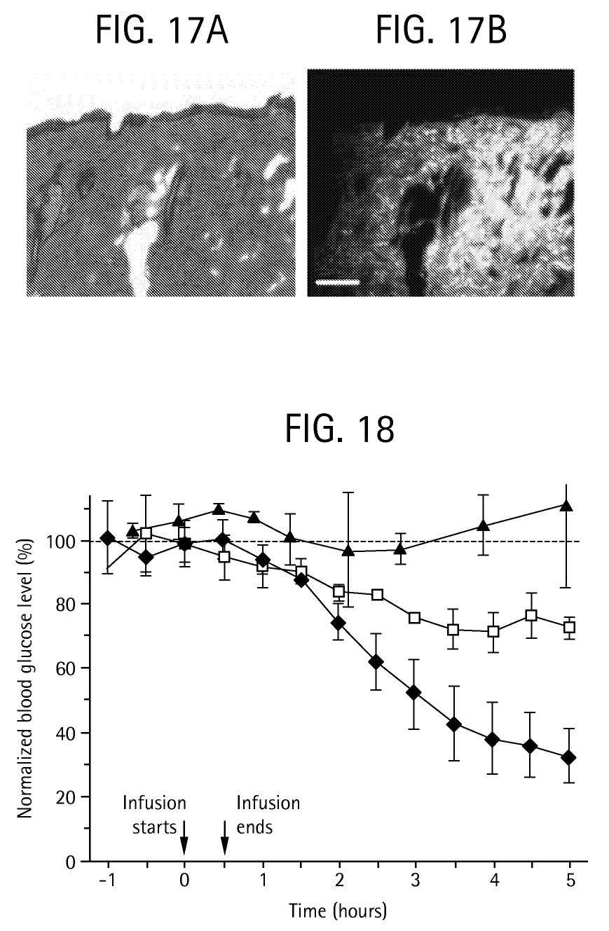

Using fluorescently labeled insulin, the drug was observed to distribute throughout the dermis (FIG. 17). Although the injection was at a specific location within the dermis, the large volume of fluid injected in this case spread throughout the dermis. Careful examination shows that there is only weak fluorescence from within the epidermis, suggesting that insulin had difficulty crossing the dermal-epidermal junction and remained within the dermis. This contrasts with microinjection of calcein, which fluoresced more uniformly in both dermis and epidermis.

As a companion experiment, insulin was microinjected into the skin of diabetic hairless rats in vivo and blood glucose concentration was monitored over time. Microinjection was carried out via microneedle insertion to a depth of 500-800 μm and infusing for 30 min. The volume of insulin solution microinjected into diabetic rat skin was 5±1 μl over the 30-min infusion period. Over a 4.5-h monitoring period after infusion, blood glucose level dropped and then plateaued at approximately 25% below pre-treatment values. This drop was significantly lower than blood glucose levels in a negative control group microinjected with saline (P<0.05). See FIG. 18, which shows (▴) infusion of saline as a negative control (n=4), (∘) infusion of insulin for microneedle inserted 800 μm (n=3), () infusion of insulin solution using microneedle inserted 800 μm and then retracted 200-300 μm (n=4). Data expressed as mean±SEM.

Recognizing that needle retraction can increase microinfusion, the insulin delivery experiment was repeated by inserting microneedles to the same initial depth in the skin and then retracting by 150-200 μm back toward the skin surface. As before, blood glucose level dropped after microinfusion, but this time dropped approximately 70% below pre-treatment values (FIG. 18), which was a significantly greater effect than that caused by saline infusion or insulin infusion without retraction (P<0.05). The volume of insulin solution microinjected into diabetic rat skin after needle retraction was 29±12 μl over the 30-min infusion period. Microinfusion in this manner may find applications for continuous or intermittent drug delivery from an indwelling microneedle, which could provide an alternative to the macroscopic catheters currently used for insulin pump therapy (Lenhard & Reeves, Arch Intern Med 161: 2293-2300 (2001)).

### EXAMPLE 6

### Delivery of Particles and Cells by Microinjection

Initial experiments to test the effects of shear stress experienced by cells during infusion through microneedles in the absence of skin flowed suspensions of newborn mouse dermal fibroblasts (provided courtesy of Dr. Kurt Stenn, Aderans Research Institute) through microneedles at rates up to 100 μl/s and showed no significant loss of cell viability.

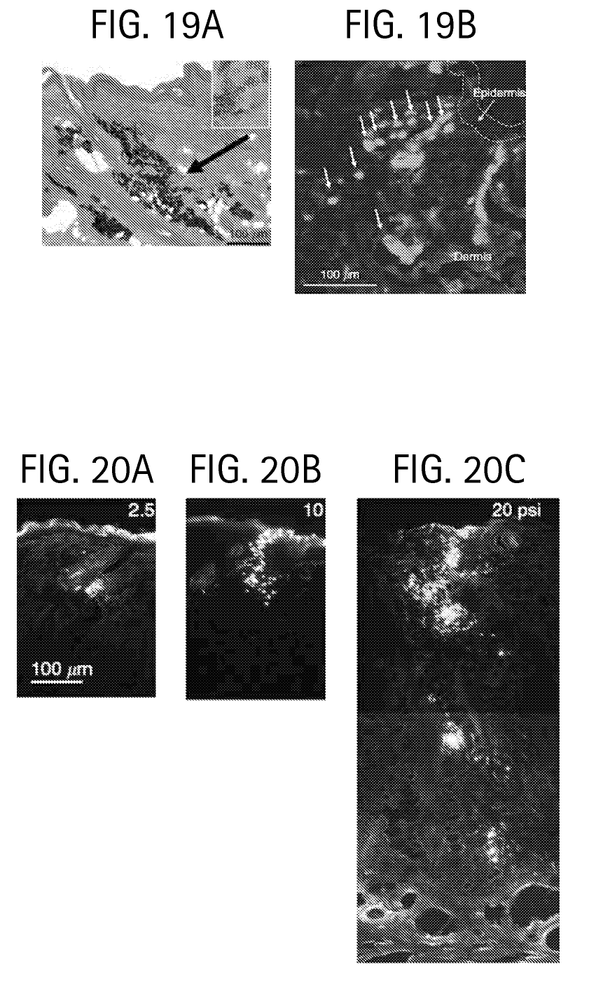

Follow-on experiments were conducted to microinject polymeric microspheres and epithelial cells into rat skin in vivo. Microneedle infusion was carried out as described in Example 4, using polymeric microparticles in PBS (2.8 μm diameter, ˜2 mg/ml, Epoxy beads M-270, Dynal Biotech, Brown Deer, Wis. or 2.5 μm diameter, 1-2% solids, carboxylate-modified FluoSpheres, yellow-green fluorescent, Molecular Probes), or Caco-2 intestinal epithelial cells (3-5×10⁶ cells/ml, American Type Culture Collection, Manassas, Va.) labeled with a fluorescent dye (Hoechst 33258, Molecular Probes). FIG. 19 demonstrates that the microparticles and cell were microinjected. At smaller injection pressures, microparticles tended to collect near the needle tip, whereas at larger pressures they filled the needle track and at still larger pressures they penetrated deeper into the skin, as illustrated in FIG. 20.

### EXAMPLE 7

### Microneedle Infusion With Vibration-Assisted Injection

Microneedle arrays were inserted into skin using vibration (without rotary drilling or retraction), using a device that vibrated the microneedle during insertion and injection into skin. The vibration motor operated at a vibration frequency of 24 Hz with an amplitude of 100 μm parallel to the needle axis. These frequency and displacement values were selected based on the convenient availability of a motor with these specifications and were not optimized. Although the oscillatory vibration amplitude during manual insertion was 100 μm, microneedles were inserted to a net displacement much deeper. Microneedle infusion was carried out as described in Example 4.

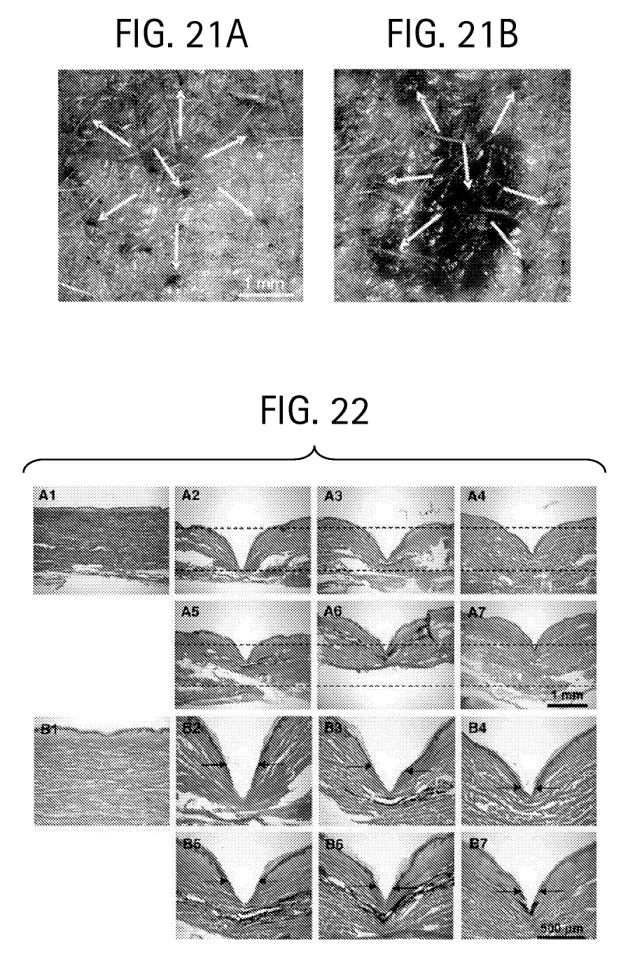

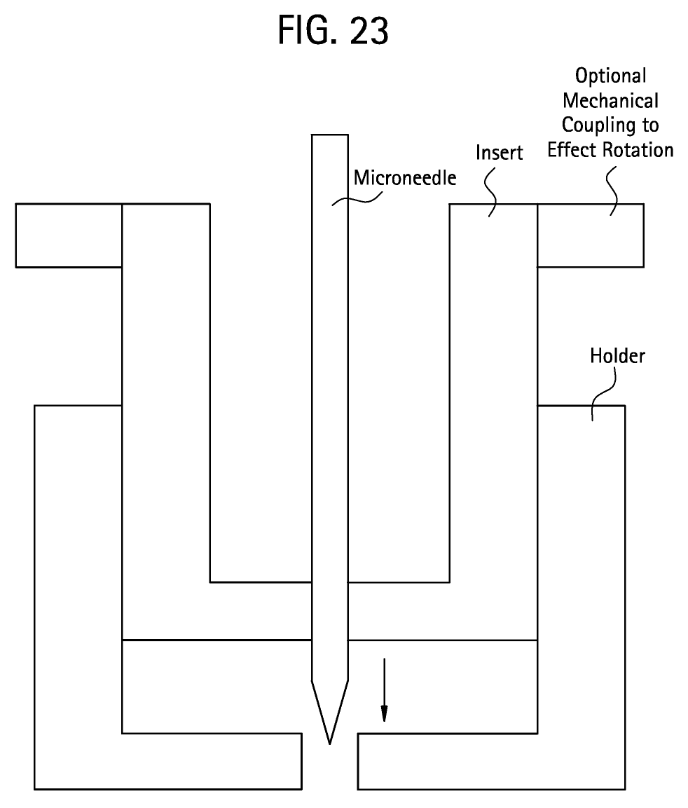

As shown in FIG. 21, injection from a seven-needle array into hairless rat skin without vibration introduced very little dye into the skin, whereas injection after insertion using vibration delivered much more, demonstrating the feasibility of a vibration-based approach.

### EXAMPLE 8

### Effect of Microneedle Insertion and Retraction on Skin Microstructure

This experiment focused on determining what happens to skin microstructure during microneedle insertion and retraction. The materials and methods used in this study were as described above in Example 4. To image infusion into the skin, a microneedle was inserted using a rotary drilling motion, as described in Example 4, to a depth of 1080 μm into the skin and either left in place or retracted 720 μm back toward the skin surface. A solution of 10⁻³ M sulforhodamine-B (Molecular Probes, Eugene, Oreg.) was then infused into the skin for 104 min at 138 kPa infusion pressure. The top surface of the skin was imaged using bright-field microscopy (Leica DC 300; Leica Microsystems, Bannockburn, Ill.).

To image microneedle insertion and retraction within skin, a microneedle was similarly inserted to a depth of 1080 μm into the skin and either left in place or retracted different distances back toward the skin surface. A small volume of blue-green dye solution (Tissue Marking Dye; Triangle Biomedical Sciences, Durham, N.C.) was then injected to mark the needle tip location. These skin samples were fixed using paraformaldehyde/glutaraldehyde with microneedles in place. Microneedles were then removed and skin samples were sectioned and examined by bright-field microscopy.

### Results

In order to better understand why microneedle retraction has such a large effect on infusion into skin, it was sought to determine what happens to skin microstructure during needle insertion. As an indirect assessment, the force applied to the skin during microneedle insertion was measured as a function of microneedle displacement using methods described previously (Davis, et al., J Biomech 37: 1155-1163 (2004)). Immediately after initial contact with the skin, the force required to move the microneedle at a microneedle displacement of 100 μm was 0.6±0.1 N. As the microneedle translated deeper, the required force steadily increased to, for example, 3.0±0.3 N at a displacement of 400 μm and 7.6±0.2 N at a displacement of 600 μm. This sharp increase in force with displacement would not be expected for piercing penetration through skin without tissue compression, which should require a relatively constant force. Indeed, force might increase as the needle penetrates deeper into skin due to increased frictional resistance caused by the increasing needle surface area contacting the skin and the increasing radius of the tapered needle that requires greater tissue displacement. However, the sharp increases in the observed force profile suggest that tissue compression also occurred due to indenting the skin surface during needle translation. This should require a continuously increasing force as compression becomes increasingly difficult while the skin is deformed from its relaxed state.

Skin mechanics during microneedle insertion can be better understood by examining skin microanatomy after insertion. In FIG. 22, skin is shown before insertion (1) or after a needle was inserted to a depth of 1080 μm and left in place (2) or partially retracted 180 μm (3), 360 μm (4), 540 μm (5), 720 μm (6), and 900 μm (7) back toward the skin surface. A small amount of blue dye was infused into the skin to mark the needle tip location and then the skin was fixed with the needle in place. Before H&E staining and histological sectioning, the microneedle was removed and is not present in the images shown. The dashed lines in (A) indicate the pre-insertion skin surface location (upper line) and the pre-retraction microneedle tip insertion depth (lower line) estimated by placing the lower line at a distance below the post-retraction needle tip location equal to the retraction distance and placing the upper line 1080 μm above the lower line. The site of needle penetration into the skin (i.e., where the stratum corneum has been breached) is indicated with arrows in (B). These images are representative of at least 3 replicates prepared at each condition.

FIG. 22A1 shows a histological cross-section of human cadaver skin before needle insertion. A thin layer of epidermis is seen atop a thick layer of dermis. FIG. 22A2 shows human cadaver skin fixed immediately after needle insertion without needle retraction. Significant indentation of the skin is seen. Examination of the magnified view in FIG. 22B2 shows that most of the indented tissue is covered by an apparently intact layer of epidermis and only the lower 100-300 μm of the tissue indentation has penetrated into the dermis (i.e., is not covered by epidermis). This suggests that during the microneedle insertion of 1080 μm, 800-1000 μm of needle displacement caused tissue indentation and only 100-300 μm caused tissue penetration.

This observation helps explain both the need for “deep” (i.e., >1 mm) microneedle insertion and the reason for the tissue compaction that it creates. Because the skin is highly elastic, pressing a microneedle against the skin initially indents the skin and only after a minimum “insertion force” is achieved does microneedle insertion occur. Thus, even though a microneedle might be displaced more than 1 mm from the initial skin surface, it only penetrates a small fraction of that distance into the skin. In this study, that fraction was approximately 10-30%. Insertion under different experimental conditions would probably lead to different depths of penetration.

This level of tissue deformation should lead to significant tissue compaction. In this study, the edges of the skin were pinned in place under tension and the skin rested on a rigid metal surface. Before microneedle insertion, skin thickness was approximately 2 mm. Skin deformation of close to 1 mm during microneedle insertion should therefore locally decrease skin thickness by ˜50%. Because cells and extracellular matrix in skin are largely fixed in position, such a large change in skin thickness would most likely squeeze out interstitial fluid. Because skin is 60-70% water (Pearce and Grimmer, J Invest Dermatol 58: 347-61 (1972)), a halving of skin thickness should require removal of almost all fluid and thereby collapse tissue porosity and effective pore size to almost zero. Changes in porosity, and especially pore size, are known to strongly affect the resistance to flow in porous materials (Jackson and James, Can J Chem Eng 64: 364-74 (1986)). Consistent with this mechanism, decreased hydration in skin has been specifically shown to reduce flow conductivity (Bert & Reed, Biorheology 32: 17-27 (1995)).

To increase the flow conductivity that was reduced during needle insertion, microneedle retraction is proposed to relieve the tissue compaction. FIGS. 22A2-22A7 show histological cross-sections of human cadaver epidermis during needle retraction from an initial insertion depth of 1080 μm (FIG. 22A2) to a final insertion depth of 180 μm (i.e., retraction of 900 μm; FIG. 22A7). During the retraction process, it appears that the microneedle remained embedded within the skin and that the retraction primarily led to a recoiling of the skin surface indentation back toward and past its original position. FIGS. 22A2-22A7 show the relative position of the microneedle penetration moving up from its initial insertion depth of 1080 μm (lower dashed line) toward the skin's original surface location (upper dashed line). The magnified images in FIGS. 22B2-22B7 show that the geometry of the site of penetration (i.e., as opposed to the site of skin deformation) remains generally unchanged during retraction. Thus, the microneedle tip appears to have remained embedded at a depth of 100-300 μm into the skin at all retraction positions. However, skin thickness beneath the needle tip appears to have steadily expanded during the retraction process, which increased skin water content and porosity/pore size, and thereby increased flow conductivity. A quantitative measure of skin thickness during retraction is difficult due to the variability in initial skin thickness between samples and the relatively small sample size (n=3 at each retraction depth).

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

1. A method of delivering a drug to a biological tissue comprising:
inserting at least one microneedle into the biological tissue;
partially retracting the at least one microneedle from the tissue; and
delivering at least one drug formulation into the biological tissue via the partially retracted at least one microneedle.

2. The method of claim 1, wherein the at least one microneedle deforms and penetrates the biological tissue during the insertion step, and the retraction step at least partially relaxes the tissue deformation while maintaining at least part of the tissue penetration.

3. The method of claim 1, wherein the at least one microneedle is rotated about its longitudinal axis during the insertion step, during the partial retraction step, or during both steps.

4. The method of claim 3, wherein the insertion and retraction steps are performed in a controlled fashion calibrated by the number of rotations of the at least one microneedle.

5. The method of claim 1, wherein the at least one microneedle is vibrated during the insertion step, during the partial retraction step, or during both steps.

6. The method of claim 1, wherein the at least one microneedle is retracted a distance that is between 10% and 90% of the initial insertion depth.

7. The method of claim 6, wherein initial insertion depth is between 200 μm and 5000 μm.

8. The method of claim 1, wherein the at least one microneedle is hollow and the drug formulation is fluid and flows through the hollow microneedle into the biological tissue.

9. The method of claim 8, wherein the fluid drug formulation is driven through the at least one microneedle in a manner effective to drive the fluid drug formulation into the biological tissue.

10. The method of claim 9, wherein the fluid drug formulation is driven by diffusion, capillary action, a mechanical pump, electroosmosis, electrophoresis, convection, magnetic field, ultrasound, or a combination thereof.

11. (canceled)

12. The method of claim 1, wherein the drug formulation comprises macromolecules, microparticles, nanoparticles, cells, viruses, or a combination thereof.

13-14. (canceled)

15. The method of claim 1, wherein the drug formulation comprises a vaccine or insulin.

16. (canceled)

17. The method of claim l, which uses an array of two or more of the microneedles.

18. The method of claim 1, wherein the biological tissue is sclera, cornea, or conjunctiva.

19. The method of claim 1, wherein the biological tissue is human skin or other mammalian skin.

20. The method of claim 19, further comprising administering at least one compound that alters the microstructure of the skin, said administering being in combination with the delivery of the at least one drug formulation.

21. The method of claim 20, wherein the at least one compound comprises hyaluronidase, collagenase, or another enzyme.

22. The method of claim 1, which is adapted to deliver the drug formulation specifically to the epidermis.

23. The method of claim 1, which is adapted to deliver the drug formulation specifically to the dermis or subcutaneous tissue.

24. A method of transdermal delivery of drug to a patient comprising:
inserting at least one microneedle into the skin of the patient;
delivering at least one drug formulation into the skin via the at least one microneedle; and
co-administering at least one compound that alters the microstructure of the skin in combination with the delivery of the at least one drug formulation.

25. The method of claim 24, wherein the at least one compound comprises hyaluronidase, collagenase, or another enzyme.

26. A method of microinfusion of a fluid into a biological tissue comprising:
inserting at least one hollow microneedle into the biological tissue; and
driving a fluid drug formulation from a fluid reservoir through the hollow microneedle into the biological tissue,
wherein deformation of the biological tissue is reduced by performing the insertion step using microneedle velocity, microneedle vibration, microneedle rotation, tissue stretching, or a combination thereof.

27. A method of fluid extraction from a biological tissue comprising:
inserting at least one hollow microneedle into the biological tissue;
partially retracting the at least one microneedle from the tissue; and
withdrawing at least one biological fluid from the biological tissue via the partially retracted at least one microneedle.

28. A microneedle device for delivery of a fluid drug formulation to or withdrawal of a fluid from a biological tissue comprising:
at least one microneedle having a tip portion for penetrating skin or another biological tissue;
means for controllably inserting the at least one microneedle into the biological tissue, deforming the biological tissue; and
means for controllably retracting the at least one microneedle partially from the tissue to at least partially relax the tissue deformation.

29. The microneedle device of claim 29, further comprising means for rotating the at least one microneedle about its longitudinal axis during the insertion step, during the partial retraction step, or during both steps.

30. The microneedle device of claim 28, further comprising means for vibrating the at least one microneedle.

31. The microneedle device of claim 28, further comprising:
a holder with a bottom surface for contacting the biological tissue;
an opening in the bottom surface allowing the at least one microneedle to pass through; and
an insert disposed inside the holder, the insert having a through bore configured to receive the at least one microneedle so positioned to pass through the opening.

32. The microneedle device of claim 31, wherein the movement of the insert along the longitudinal axis is effectuated by a mechanical coupling element attached to the insert.

33. The microneedle device of claim 32, wherein said mechanical coupling element comprises a gear for coupling to another gear, a motor, or a micromotor, or may be manually driven.

34. The microneedle device of claim 28, further comprising a fluid reservoir storing at least one fluid drug formulation to be delivered into the biological tissue.

35. The microneedle device of claim 34, wherein the drug formulation comprises macromolecules, microparticles, nanoparticles, cells, or a combination thereof.

36-37. (canceled)

38. The microneedle device of claim 34, wherein the drug formulation comprises hyaluronidase.

39-41. (canceled)

42. The microneedle device of claim 28, wherein the means for controllably inserting the microneedle is adapted to provide a maximum insertion depth into the biological tissue between 200 μm and 5000 μm.

43. The microneedle device of claim 42, wherein the means for controllably inserting the microneedle is adapted to provide a maximum insertion depth into the biological tissue between 500 and 1500 μm.

44. The microneedle device of claim 28, further comprising at least one pump device for pumping the drug from the fluid source to the tip end of the microneedle.
